(19) United States
(12) Patent Application Publication
(10) Pub. No.: US 2022/0297096 A1
(43) Pub. Date: Sep. 22, 2022

(54) NITROGEN CONTAINING BIOPOLYMER-BASED CATALYSTS, A PROCESS FOR THEIR PREPARATION AND USES THEREOF

(71) Applicant: Hoffmann-La Roche Inc., Little Falls, NJ (US)

(72) Inventors: Stephan BACHMANN, Allschwil (CH); Matthias BELLER, Rostock (DE); Dario FORMENTI, Milano (IT); Kathrin JUNGE, Rostock (DE); Basudev SAHOO, Rostock (DE); Michelangelo SCALONE, Birsfelden (CH); Christoph TOFP, Linz (AT)

(73) Assignee: Hoffmann-La Roche Inc., Little Falls, NJ (US)

(21) Appl. No.: 17/702,043

(22) Filed: Mar. 23, 2022

Related U.S. Application Data
(62) Division of application No. 16/446,282, filed on Jun. 19, 2019.
Application No. 16/446,282 is a continuation of application No. PCT/EP2017/083276, filed on Dec. 18, 2017.

(30) Foreign Application Priority Data
Dec. 19, 2016 (EP) 16002691.0

(51) Int. Cl. B01J 23/755; B01J 21/18; B01J 23/34; B01J 23/52; B01J 23/72; B01J 23/745; B01J 23/75; B01J 35/02; B01J 37/04; B01J 37/08; C07B 35/06

(52) U.S. Cl. CPC B01J 23/755; B01J 21/18; B01J 23/34; B01J 23/52; B01J 23/72; B01J 23/745; B01J 23/75; B01J 35/026; B01J 37/04; B01J 37/086; C07B 35/06

(57) ABSTRACT

The present invention relates to a novel process for the preparation of a nitrogen containing biopolymer-based catalyst and to the novel nitrogen containing biopolymer-based catalysts obtainable by this process. In particular, the invention relates to a novel nitrogen containing biopolymer-based catalyst comprising metal particles and at least one nitrogen containing carbon layer. The invention also relates to the use of a nitrogen containing biopolymer-based catalyst in a hydrogenation process, preferably in a process for hydrogenation of nitroarenes, nitriles or imines; in a reductive dehalogenation process of C—X bonds, wherein X is Cl, Br or I, preferably in a process for dehalogenation of organohalides or in a process for deuterium labelling of arenes via dehalogenation of organohalides; or in an oxidation process. Further, the invention relates to a metal complex with the nitrogen containing biopolymer, wherein the metal is a transition metal selected from the group consisting of manganese, ruthenium, cobalt, rhodium, nickel, palladium and platinum, and wherein the nitrogen containing biopolymer is selected from chitosan, chitin and a polyamino acid.

### CROSS REFERENCE TO RELATED APPLICATIONS

This application is a divisional of U.S. application Ser. No. 16/446,282, filed on Jun. 19, 2019, which is a continuation of International Application No. PCT/EP2017/083276, filed on Dec. 18, 2017, which claims priority to EP Application No. 16002691.0, filed on Dec. 19, 2016, the disclosures of which are incorporated herein by reference in their entireties.

### FIELD OF THE INVENTION

The present invention relates to a novel process for the preparation of a nitrogen containing biopolymer-based catalyst and to the novel nitrogen containing biopolymer-based catalysts obtainable by this process. In particular, the invention relates to a novel nitrogen containing biopolymer-based catalyst comprising metal particles and at least one nitrogen containing carbon layer. The invention also relates to the use of a nitrogen containing biopolymer-based catalyst in a hydrogenation process, preferably in a process for hydrogenation of nitroarenes, nitriles or imines; in a reductive dehalogenation process of C—X bonds, wherein X is Cl, Br or I, preferably in a process for dehalogenation of organohalides or in a process for deuterium labelling of arenes via dehalogenation of organohalides; or in an oxidation process. Further, the invention relates to a metal complex with the nitrogen containing biopolymer, wherein the metal is a transition metal selected from the group consisting of manganese, ruthenium, cobalt, rhodium, nickel, palladium and platinum, and wherein the nitrogen containing biopolymer is selected from chitosan, chitin and a polyamino acid.

### BACKGROUND OF THE INVENTION

Hydrogenation catalysts are widely used for the preparation of intermediate compounds required for the synthesis of various chemical compounds. Most frequently, industrial hydrogenation relies on heterogeneous catalysts.

U.S. Pat. No. 8,658,560 B1 describes a hydrogenation catalyst for preparing aniline from nitrobenzene, which contains palladium and zinc on a carrier.

US 2012/0065431 A1 proposes the preparation of aromatic amines by catalytically hydrogenating the corresponding aromatic nitro compounds using a copper catalyst with a support comprising silicon dioxide (SiO₂). The preparation of the catalyst requires the preparation of SiO₂ by wet grinding and subsequent spray drying.

US 2004/0176619 A1 describes the use of ruthenium catalysts on amorphous silicon dioxide as support material for the preparation of sugar alcohols by catalytic hydrogenation of the corresponding carbohydrates.

WO 02/30812 A2 describes a hydrodehalogenation process using a catalyst containing nickel on aluminum oxide as support material.

Thus, there is a need for novel alternative catalysts, which are suitable for use in a hydrogenation process, for example in a process for the hydrogenation of nitroarenes, nitriles or imines; in a reductive dehalogenation process of C—X bonds, wherein X is Cl, Br or I, preferably in a process for dehalogenation of organohalides or in a process for deuterium labelling of arenes via dehalogenation of organohalides; or in an oxidation process. In particular, the need exists for catalysts, preferably for hydrogenation catalysts having a high metal content and large nitrogen content. Furthermore, hydrogenation catalysts are of interest, which can be used without any additional support materials such as silicon dioxide, aluminium oxide or carbon.

### SUMMARY OF THE INVENTION

The present invention, in one aspect, relates to a process for the preparation of a nitrogen containing biopolymer-based catalyst comprising the steps of:
(a) mixing a metal precursor in the presence of a solvent with a nitrogen containing biopolymer to obtain a metal complex with the nitrogen containing biopolymer;
(b) if appropriate drying the metal complex with the nitrogen containing biopolymer; and
(c) pyrolysing the metal complex with the nitrogen containing biopolymer at temperatures ranging from 500° C. to 900° C. in an inert gas atmosphere to obtain a nitrogen containing biopolymer-based catalyst.

In one embodiment, in the process of the invention, the metal precursor contains a transition metal.

In another embodiment, in the process of the invention, the metal precursor contains a transition metal selected from the group consisting of manganese, iron, ruthenium, cobalt, rhodium, nickel, palladium, platinum and copper.

In a preferred embodiment, in the process of the invention, the metal precursor contains a transition metal selected from the group consisting of manganese, iron, cobalt, nickel and copper. Particularly preferred transition metals are cobalt or nickel more preferably cobalt

In another embodiment, in the process of the invention, the metal precursor is a metal salt, preferably selected from the group consisting of acetate, bromide, chloride, iodide, hydrochloride, hydrobromide, hydroiodide, hydroxide, nitrate, nitrosylnitrate and oxalate salts, or a metal chelate, preferably an acetylacetonate chelate.

In another embodiment, in the process of the invention, the solvent is selected from the group consisting of alcohols, preferably ethanol, and water, or mixtures thereof.

In another embodiment, the nitrogen containing biopolymer is selected from chitosan, chitin, or a polyamino acid. Particularly preferred nitrogen containing biopolymers are chitosan or chitin, preferably chitosan.

In another embodiment, in the process of the invention, the metal complex with the nitrogen containing biopolymer is pyrolysed at temperatures ranging from 550° C. to 850° C., preferably at temperatures ranging from 600° C. to 800° C.

In another embodiment, in the process of the invention, pyrolysis time ranges from 10 minutes to three hours, preferably pyrolysis time ranges from one hour to two hours.

In another aspect, the present invention relates to a nitrogen containing biopolymer-based catalyst obtainable according to the process as defined herein.

In another aspect, the present invention relates to a nitrogen containing biopolymer-based catalyst comprising metal particles and at least one nitrogen containing carbon layer.

In one embodiment, the metal particles comprise metallic and/or oxidic metal particles, preferably metallic and/or oxidic manganese, iron, ruthenium, cobalt, rhodium, nickel, palladium, platinum or copper particles.

In a preferred embodiment, the metal particles comprise metallic and/or oxidic manganese, iron, cobalt, nickel or copper particles.

In a particular preferred embodiment, the metal particles are metallic and/or oxidic cobalt or nickel particles, even more preferred cobalt particles.

In one embodiment, the nitrogen containing biopolymer-based catalyst comprises from 2 to 100 nitrogen containing carbon layers.

In one embodiment, the nitrogen containing carbon layers comprise graphitic nitrogen, pyridinic nitrogen and/or pyrrolic nitrogen.

In one embodiment, the metal content of the nitrogen containing biopolymer-based catalyst ranges from 0.5 wt % to 20 wt %.

In another aspect, the present invention relates to the use of a nitrogen containing biopolymer-based catalyst in a hydrogenation process, preferably in a process for hydrogenation of nitroarenes, nitriles or imines; in a reductive dehalogenation process of C—X bonds, wherein X is Cl, Br or I, preferably in a process for dehalogenation of organohalides or in a process for deuterium labelling of arenes via dehalogenation of organohalides; or in an oxidation process.

In another aspect, the present invention relates to a method of hydrogenation, a method of reductive dehalogenation of C—X bonds, wherein X is Cl, Br or I, or a method of oxidation, conducted in the presence of a nitrogen containing biopolymer-based catalyst as defined herein.

In one embodiment, the method of hydrogenation comprises the step of contacting a nitroarene, a nitrile or an imine with hydrogen gas in the presence of a nitrogen containing biopolymer-based catalyst as defined herein.

In one embodiment, the method of reductive dehalogenation comprises the step of contacting an organohalide with hydrogen gas in the present of a nitrogen containing biopolymer-based catalyst as defined herein.

In another aspect, the present invention relates to a metal complex with the nitrogen containing biopolymer, wherein the metal is a transition metal selected from the group consisting of manganese, ruthenium, cobalt, rhodium, nickel, palladium, platinum and copper, and wherein the nitrogen containing biopolymer is selected from chitosan, chitin and a polyamino acid.

In a preferred embodiment, in the metal complex of the invention, the metal is cobalt(II) or nickel(II) and the nitrogen containing biopolymer is selected from chitosan, chitin or a polyamino acid. Preferably, the nitrogen containing biopolymer is chitosan or chitin, more preferably chitosan.

Any combinations of any embodiments of the different aspects of the present invention as defined herein, e.g. of the process for the preparation of a nitrogen containing biopolymer-based catalyst, of the nitrogen containing biopolymer-based catalyst, of the use of the nitrogen containing biopolymer-based catalyst, of the methods of hydrogenation and oxidation and of the metal complex with the nitrogen containing biopolymer are considered to be within the scope of the invention.

### BRIEF DESCRIPTION OF THE FIGURES

FIGS. 1A-1F show high resolution scanning transmission electron microscopy (STEM) images of the CoOₓ@Chit-700 catalyst; FIG. 1A, FIG. 1B, FIG. 1C, FIG. 1E, and FIG. 1F show annular bright field (ABF) images of the CoOₓ@Chit-700 catalyst. FIG. 1D shows high-angle annular dark field (HAADF) images of cobalt composites of the CoOₓ@Chit-700 catalyst.

FIG. 2A, FIG. 2C, FIG. 2D, FIG. 2E and FIG. 2F show energy-dispersive X-ray spectroscopy (EDXS) images of the CoOₓ@Chit-700 catalyst. FIG. 2B shows a high resolution ABF (HR-ABF) image of the CoOₓ@Chit-700 catalyst.

FIGS. 3A-3C show XPS spectra of the CoOₓ@Chit-700 catalyst. FIG. 3A shows a C1s XPS spectrum. FIG. 3B shows a N1s xPS spectrum; and FIG. 3C shows a Co2p XPS spectrum.

FIG. 4A and FIG. 4B show X-ray photoelectron spectroscopy (XPS) comparison spectra of pure chitosan.

FIG. 5 shows an X-ray diffraction (XRD) spectrum of the CoOₓ@Chit-700 catalyst.

FIG. 6 shows the yields and selectivity of hydrogenation of nitroarenes with the CoOₓ@Chit-700 catalyst after 1 to 5 runs.

### DETAILED DESCRIPTION OF THE INVENTION

### Novel Process for the Preparation of a Nitrogen Containing Biopolymer-Based Catalyst and Novel Nitrogen-Containing Biopolymer-Based Catalysts Obtainable According to Said Process

As indicated above, there is a need for novel alternative catalysts, which are suitable for use in a hydrogenation process, for example in a process for the hydrogenation of nitroarenes, nitriles or imines; in a reductive dehalogenation process of C—X bonds, wherein X is Cl, Br or I, preferably in a process for dehalogenation of organohalides or in a process for deuterium labelling of arenes via dehalogenation of organohalides; or in an oxidation process. In particular, the need exists for catalysts, preferably for hydrogenation catalysts, having a high metal content and large nitrogen content. Furthermore, catalysts, preferably hydrogenation catalysts are of interest, which can be used without any additional support materials such as silicon dioxide or carbon.

A problem of the present invention was therefore to provide novel alternative catalysts, preferably hydrogenation catalysts, having the above-mentioned desired characteristics.

In one aspect, the present invention provides a novel process for the preparation of a nitrogen containing biopolymer-based catalyst comprising the steps of:
(a) mixing a metal precursor in the presence of a solvent with a nitrogen containing biopolymer to obtain a metal complex with the nitrogen containing biopolymer;
(b) if appropriate drying the metal complex with the nitrogen containing biopolymer; and
(c) pyrolysing the metal complex with the nitrogen containing biopolymer at temperatures ranging from 500° C. to 900° C. in an inert gas atmosphere to obtain a nitrogen containing biopolymer-based catalyst.

The metal precursor used as a starting material in process step (a) is commercially available and contains a transition metal.

In one embodiment, the transition metal is selected from the group consisting of manganese, iron, ruthenium, cobalt, rhodium, nickel, palladium, platinum and copper. In a preferred embodiment, the transition metal is selected from the group consisting of manganese, iron, cobalt, nickel and copper. This selection addresses the particular need to develop catalysts with non-noble metals. Particularly preferred transition metals are cobalt or nickel, but more preferably cobalt.

In one embodiment, the metal precursor is a metal salt, preferably selected from the group consisting of acetate, bromide, chloride, iodide, hydrochloride, hydrobromide, hydroiodide, hydroxide, nitrate, nitrosylnitrate and oxalate salts, or a metal chelate, preferably an acetylacetonate chelate.

In a preferred embodiment, the metal salts, which are used as starting material in process step (a) include but are not limited to Co(OAc)₂.4H₂O, Co(NO₃)₂, Co(OH)₂, Fe(OAc)₂, Cu(acac)₂, Ni(OAc)₂.4H₂O and MnCl₂. In a particular preferred embodiment, Co(OAc)₂.4H₂O, Co(NO₃)₂ or Co(OH)₂ are used as starting material in process step (a). The most preferred metal salts are Co(OAc)₂.4H₂O or Ni(OAc)₂.4H₂O.

The nitrogen containing biopolymer used as a starting material in process step (a) is commercially available and includes but is not limited to chitosan, chitin and polyamino acids, such as polylysine.

In one embodiment, the nitrogen containing biopolymer used as a starting material in process step (a) is commercially available and is based on chitosan or on chitin, preferably on chitosan.

Suitable chitosan is commercially available low molecular weight chitosan having a molecular weight ranging from 50,000 to 190,000 Da and a viscosity of 20 to 300 cP (1 wt % in 1% acetic acid, 25° C., Brookfield).

Another suitable chitosan is commercially available medium molecular weight chitosan having a viscosity of 200 to 800 cP (1 wt % in 1% acetic acid, 25° C., Brookfield).

Another suitable chitosan is commercially available high molecular weight chitosan having a molecular weight ranging from 310,000 to 375,000 Da having a viscosity of 800 to 2000 cP (1 wt % in 1% acetic acid, 25° C., Brookfield).

In a preferred embodiment, shrimp shell derived chitosan is used as a starting material.

For carrying out process step (a), in general from 5 mmol to 10 mmol chitosan, preferably from 6 mmol to 9 mmol chitosan, particularly preferred from 6 mmol to 9 mmol of chitosan are employed per mmol metal precursor.

In a preferred embodiment, 8.6 mmol chitosan are employed per mmol Co(OAc)₂.4H₂O.

Suitable solvents for carrying out process step a) are alcohols such as methanol, ethanol, n- or i-propanol, n-, i-, sec- or tert-butanol, ethanediol, propane-1,2-diol, ethoxyethanol, methoxyethanol, diethylene glycol monomethyl ether, diethylene glycol monoethyl ether, mixtures thereof with water, or water. In a preferred embodiment, ethanol is used as a solvent.

For carrying out process step (a), in general, from 10 mL to 70 mL solvent per mmol of metal precursor are employed, e.g. from 20 mL to 60 mL solvent per mmol of metal precursor, or from 30 mL to 50 mL solvent per mmol of metal precursor.

When carrying out process step a), the reaction temperatures can be varied within a relatively wide range. In general, process step (a) is carried out at temperatures ranging from room temperature to 90° C., e.g. from 30° C. to 80° C., from 40° C. to 75° C., or from 50° C. to 70° C., preferably at 70° C.

When carrying out process step a), the suspension is stirred for 2 hours to 20 hours, e.g. for 2 hours to 18 hours, for 3 hours to 16 hours, for 4 hours to 10 hours, or for 4 hours to 6 hours, preferably for 4 hours.

In a preferred embodiment of the process of the invention, the metal complex with the nitrogen containing biopolymer, preferably the metal complex with chitosan or chitin more preferably chitosan, which is obtained according to process step (a), is dried in process step (b) by customary techniques, preferably under vacuum.

When carrying out process step (c), in general, the metal complex with the nitrogen containing biopolymer, preferably the metal complex with chitosan or chitin more preferably chitosan, is pyrolysed at temperatures ranging from 500° C. to 900° C., e.g. from 550° C. to 850° C., from 600° C. to 800° C., from 650° C. to 750° C., at 600° C., at 700° C. or at 800° C. to obtain the nitrogen containing biopolymer-based catalyst, preferably the chitosan- or chitin-based catalyst. In a particular preferred embodiment, the nitrogen containing biopolymer-based catalyst, preferably the chitosan-based catalyst, is pyrolysed at 700° C.

When carrying out process step (c), in general, the pyrolysis time ranges from 10 minutes to 3 hours, e.g. from 20 minutes to 2.5 hours, e.g. from 40 minutes to 2 hours.

In a preferred embodiment of process step (c), pyrolysis is carried out under argon atmosphere.

In general, process steps (a) and (c) are carried out under atmospheric pressure. However, it is also possible to operate under elevated or reduced pressure, in general between 10 kPa (0.1 bar) and 1000 kPa (10 bar).

The process of the invention is generally carried out according to the following procedure: The metal salt is dissolved in the solvent. Then, commercially available nitrogen containing biopolymer, preferably chitosan or chitin, particularly preferred shrimp shell derived chitosan with low viscosity, is added and the so-obtained suspension is stirred at 70° C. to obtain a metal complex with the nitrogen containing biopolymer, preferably a metal complex with the chitosan or chitin, particularly preferred a metal complex with shrimp shell derived chitosan with low viscosity (process step (a)).

Subsequently, the solvent is removed by slow rotary evaporation and the remaining solid metal complex with the nitrogen containing biopolymer, preferably a metal complex with the chitosan or chitin, particularly preferred a metal complex with shrimp shell derived chitosan with low viscosity is dried at 60° C. under vacuum to yield a dried metal complex with the nitrogen containing biopolymer, preferably a dried metal complex with the chitosan or chitin, particularly preferred a dried metal complex with shrimp shell derived chitosan (process step (b)).

Finally, the dried metal complex with the nitrogen containing biopolymer, preferably a dried metal complex with the chitosan or chitin, particularly preferred a dried metal complex with shrimp shell derived chitosan is transferred into a crucible equipped with a lid and pyrolysed at temperatures ranging from 500° C. to 900° C. under an Ar atmosphere to obtain the nitrogen containing biopolymer-based catalyst of the invention, preferably the chitosan- or chitin-based catalyst of the invention, particularly preferred the shrimp shell derived chitosan-based catalyst of the invention (process step (c)).

The process of the invention may be carried out e.g. as shown in Scheme 1 below.

It is extremely surprising that the process of the invention yields nitrogen containing biopolymer-based catalysts, preferably chitosan-based catalysts, particularly preferred shrimp shell derived chitosan-based catalysts having a high metal content and also large nitrogen content.

Moreover, unexpectedly, the nitrogen containing biopolymer-based catalysts, preferably the chitosan-based catalysts, comprise metallic and/or oxidic metal particles.

Furthermore, it has been unexpectedly found that the metallic metal particles are partially enveloped by oxidic metal within a matrix of graphitic carbon. Consequently, due to said matrix of graphitic carbon, the process of the invention yields nitrogen containing biopolymer-based catalysts, preferably chitosan- or chitin-based catalysts, more preferably chitosan, which can be used without any additional support materials.

Thus, in another aspect, the invention relates to a nitrogen containing biopolymer-based catalyst, preferably to a chitosan- or chitin-based catalyst obtainable according to the process described herein.

Thus, in another aspect, the present invention relates to a nitrogen containing biopolymer-based catalyst comprising metal particles and at least one nitrogen containing carbon layer. In a preferred embodiment, the invention relates to a chitosan- or chitin-based catalyst. More preferred to a chitosan based catalyst. In the nitrogen containing biopolymer-based metal particles, preferably metal nanoparticles are in contact with at least one nitrogen containing carbon layer.

In one embodiment, the metal particles comprise metallic and/or oxidic metal particles, preferably metallic and/or oxidic manganese, iron, ruthenium, cobalt, rhodium, nickel, palladium, platinum and copper particles. In a preferred embodiment, the metal particles comprise metallic and/or oxidic manganese, iron, cobalt, nickel and copper particles, more preferred cobalt or nickel particles. In a particular preferred embodiment, the metal particles are metallic and/or oxidic cobalt particles.

In one embodiment, the nitrogen containing biopolymer-based catalyst comprises from 2 to 100 nitrogen containing carbon layers, e.g. from 2 to 80 nitrogen containing carbon layers, from 2 to 50 nitrogen containing carbon layers, from 5 to nitrogen containing carbon layers. In a preferred embodiment, the nitrogen containing biopolymer-based catalyst comprises from 5 to 30 nitrogen containing carbon layers.

In one embodiment, the nitrogen containing carbon layers comprise graphitic nitrogen, pyridinic nitrogen and/or pyrrolic nitrogen.

In one embodiment, the metal content of the nitrogen containing biopolymer-based catalyst ranges from 0.5 wt % to 20 wt % based on the total weight of the nitrogen containing biopolymer-based catalyst, e.g. from 3 wt % to 20 wt %, from 5 wt % to 15 wt %, or from 6 wt % to 15 wt %. With the preferred cobalt particles the content preferably ranges from 6 wt % to 12 wt % with nickel particles the content ranges from 8 wt % to 15 wt %.

The composition of the chitosan-based catalysts of the invention which may be obtained at pyrolysis temperatures of 600° C., 700° C., 800° C. and 900° C., may be determined by elemental analysis and is shown in Table 1a below.

TABLE 1a
Composition of chitosan-based catalysts of the invention

| | Pyrolysis | | | | |
|---|---|---|---|---|---|
| | temper- | | | | |
| | ature | C | H | N | Co |
| Catalyst | (° C. ) | (wt %) | (wt %) | (wt %) | (wt %) |
| CoOₓ@Chit-600 | 600 | 70.16 | 1.14 | 6.65 | 8.44 |
| CoOₓ@Chit-700 | 700 | 73.78 | 0.60 | 3.23 | 9.76 |
| CoOₓ@Chit-800 | 800 | 78.81 | 0.69 | 3.19 | 9.32 |
| CoOₓ@Chit-900 | 900 | 79.10 | 0.15 | 3.09 | 10.49 |

The composition of the chitin-based catalysts of the invention which may be obtained at pyrolysis temperatures of 700° C. and 800° C., may be determined by elemental analysis and is shown in Table 1 b below

TABLE 1b
Composition of chitosan-based catalysts of the invention

| | Pyrolysis | | | | |
|---|---|---|---|---|---|
| | temper- | | | | |
| | ature | C | H | N | Co |
| Catalyst | (° C. ) | (wt %) | (wt %) | (wt %) | (wt %) |
| CoOₓ@Chitin-700 | 700 | 70.56 | 0.264 | 2.326 | 11.783 |
| CoOₓ@Chitin-800 | 800 | 74.04 | 0.165 | 2.02 | 11.356 |
| NiOₓ@Chitin-700 | 700 | 68.69 | 0.495 | 5.052 | 13.381 |
| NiOₓ@Chitin-800 | 800 | 68.45 | 0.350 | 3.403 | 14.266 |

Metal complexes with the nitrogen containing biopolymer, wherein the metal is a transition metal selected from the group consisting of manganese, ruthenium, cobalt, rhodium, nickel, palladium, platinum and copper, may be obtained by process step (a) of the process of the invention. These metal chitosan- or chitin-complexes are novel and are also subject-matter of the invention.

Thus, in another aspect, the present invention relates to a metal complex with the nitrogen containing biopolymer, wherein the metal is a transition metal selected from the group consisting of manganese, ruthenium, cobalt, rhodium, nickel, palladium platinum and copper, preferably cobalt or nickel, more preferably cobalt, and wherein the nitrogen containing biopolymer is selected from chitosan, chitin and a polyamino acid, preferably chitosan or chitin more preferably chitosan.

In one embodiment, in the metal complex of the invention, the metal is cobalt(II) and the nitrogen containing biopolymer is selected from chitosan, chitin and a polyamino acid, preferably chitosan or chitin, more preferably chitosan.

In a preferred embodiment, the nitrogen containing biopolymer-based catalyst is a cobalt(II) chitosan or chitin or a nickel(II) chitin or chitosan complex, more preferably a cobalt(II) chitosan complex.

### Use of the Novel Nitrogen Containing Biopolymer-Based Catalysts

Furthermore, it has been found that the nitrogen containing biopolymer-based catalysts of the invention are suitable for use in a hydrogenation process. The chitosan- or chitin-based catalysts of the invention have been found to be particularly suitable for the hydrogenation of nitroarenes, nitriles or imines.

Moreover, it has been found that the nitrogen containing biopolymer-based catalysts of the invention are suitable for use in a reductive dehalogenation process of C—X bonds, wherein X is Cl, Br or I. The chitosan- or chitin-based catalysts of the invention have been found to be particularly suitable for a process for dehalogenation of organohalides or in a process for deuterium labelling of arenes via dehalogenation of organohalides.

In addition, it has been found that the nitrogen containing biopolymer-based catalysts of the invention are suitable for use in an oxidation process.

Thus, in another aspect, the present invention relates to the use of a nitrogen containing biopolymer-based catalyst in a hydrogenation process, preferably in a process for hydrogenation of nitroarenes, nitriles or imines; in a reductive dehalogenation process of C—X bonds, wherein X is Cl, Br or I, preferably in a process for dehalogenation of organohalides or in a process for deuterium labelling of arenes via dehalogenation of organohalides; or in an oxidation process.

In another aspect, the present invention relates to a method of hydrogenation, a method of reductive dehalogenation of C—X bonds, wherein X is Cl, Br or I, or a method of oxidation, conducted in the presence of a nitrogen containing biopolymer-based catalyst as defined herein.

In one embodiment, the method of hydrogenation comprises the step of reacting a nitroarene, a nitrile or an imine with hydrogen gas in the presence of a nitrogen containing biopolymer-based catalyst as defined herein.

In one embodiment, the method of reductive dehalogenation comprises the step of reacting an organohalide with hydrogen gas in the present of a nitrogen containing biopolymer-based catalyst as defined herein.

### Use of the Novel Nitrogen Containing Biopolymer-Based Catalysts in a Hydrogenation Process

In a preferred embodiment, the invention relates to the use of a chitosan- or chitin-based catalyst in a hydrogenation process.

Hydrogenation processes vary from practitioner to practitioner. It is believed that the nitrogen containing biopolymer-based catalysts, preferably the chitosan-based catalysts of the invention are applicable to all specific types of hydrogenation processes.

The nitrogen containing biopolymer-based catalysts, preferably the chitosan- or chitin-based catalysts are not to be limited by the description of the processes of using same, as described herein.

In general, the hydrogenation process is carried out at superatmospheric hydrogen pressure, e.g. at a hydrogen partial pressure of at least 1000 kPa (10 bar), preferably at least 2000 kPa (20 bar) and in particular at least 4000 kPa (40 bar). In general, the hydrogen partial pressure will not exceed a value of 50000 kPa (500 bar), in particular 35000 kPa (350 bar). The hydrogen partial pressure ranges particularly preferred from 4000 kPa (40 bar) to 20000 kPa (200 bar). The hydrogenation reaction is generally carried out at temperatures of at least 40° C. In particular, the hydrogenation process is carried out at temperatures ranging from 80° C. to 150° C.

The process conditions of hydrogenation processes are well known to the skilled person.

### Hydrogenation of Nitroarenes

In one embodiment, a nitrogen containing biopolymer-based catalyst, preferably a chitosan- or chitin-based catalyst of the invention as defined herein is used in a process for hydrogenation of nitroarenes, in particular for preparing aniline from nitrobenzene, or for preparing substituted anilines from the respective substituted nitrobenzene.

In one aspect, the present invention relates to a method for preparing an aromatic amino compound, comprising the step of reacting a nitroarene with hydrogen gas in the presence of a nitrogen containing biopolymer-based catalyst, preferably a chitosan- or chitin-based catalyst of the invention as defined herein. Furthermore, the nitrogen containing biopolymer-based catalyst, preferably the chitosan- or chitin-based catalyst is suitable for the preparation of any aromatic amino compounds from the nitro compounds, e.g. of intermediates of any kind of products, e.g. of pharmaceutical drugs or of plant protection products. The nitrogen containing biopolymer-based catalyst, preferably the chitosan- or chitin-based catalyst may also be used directly for the preparation of pharmaceutical drugs or pesticides.

As used herein, the term “nitroarenes” comprise substituted and unsubstituted nitroarenes.

Scheme 2 illustrates the conversion ratios and reaction times of substituted nitroarenes when reacting the substituted nitroarenes with a nitrogen containing biopolymer-based catalyst, preferably a chitosan- or chitin-based catalyst of the invention, e.g. with the Co—Co₃Co₄@Chit-700 catalyst of the invention. As shown in Scheme 2, substituted nitroarenes may be hydrogenated in the presence of hydrogen gas, the Co—Co₃Co₄@Chit-700 catalyst of the invention and triethylamine in a mixture of ethanol and water.

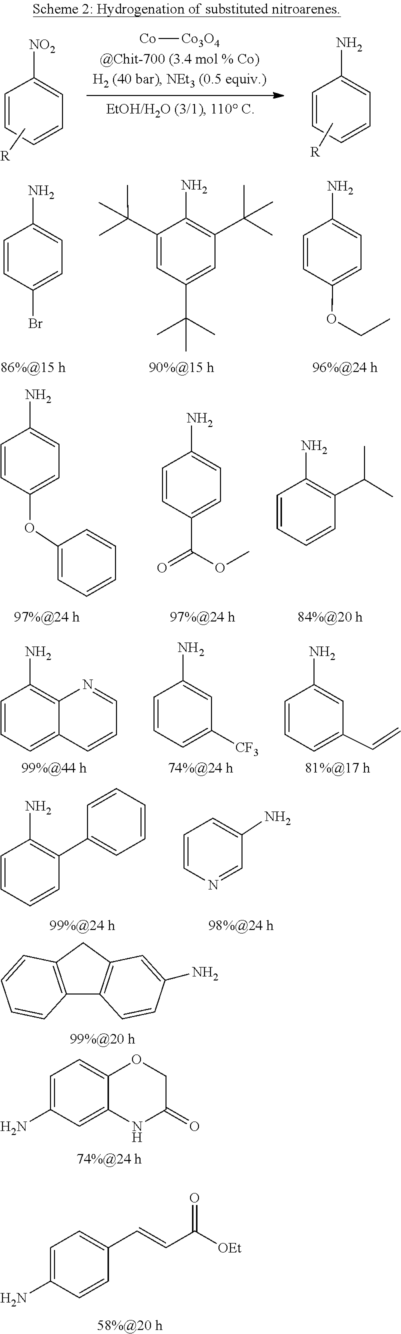

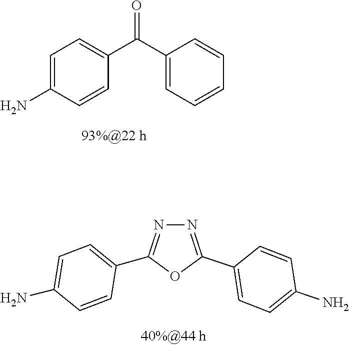 [SMILES: Nc1ccc(C(=O)c2ccccc2)cc1 ; Nc1ccc(-c2nnc(-c3ccc(N)cc3)o2)cc1]

For example, pharmaceutical drugs may be obtained by hydrogenation of the nitroarenes nimesulide and flutamide.

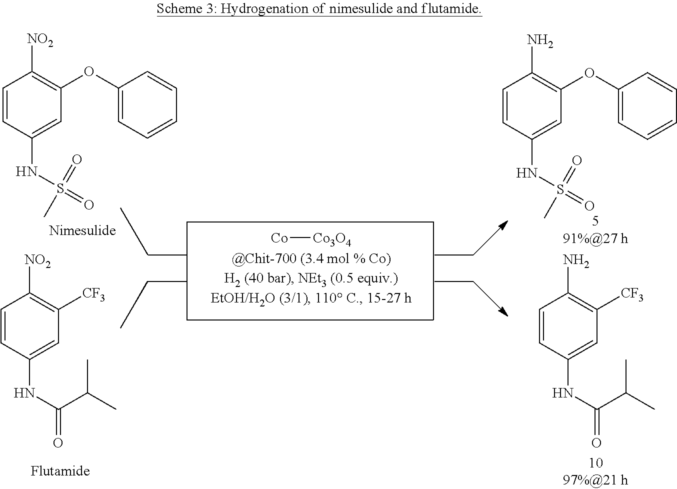

Furthermore, it has been surprisingly found that the selectivity of the hydrogenation of nitrobenzene with the CoOₓ@Chit-700 catalyst of the invention under the reaction conditions depicted in Scheme 4 is constant over 5 runs.

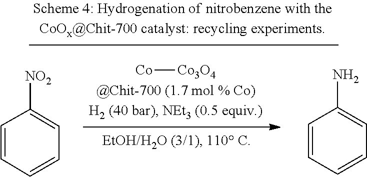

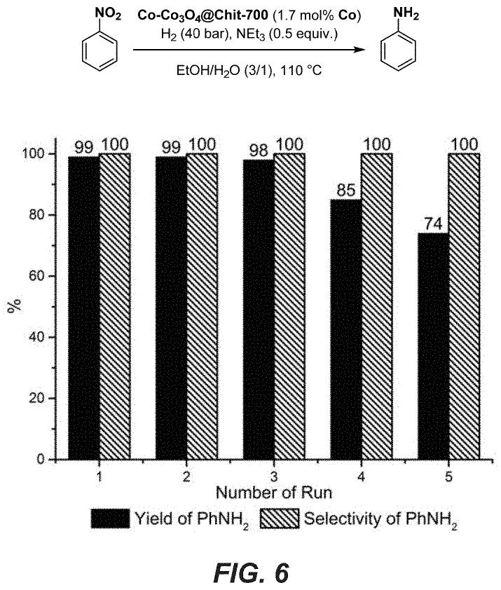

The results of these recycling experiments of hydrogenation of nitrobenzene are summarized in the bar graph of FIG. 6. FIG. 6 shows the yields and selectivity of hydrogenation of nitrobenzene with the CoOₓ@Chit-700 catalyst after 1 to 5 runs. It has been found that the yield of the hydrogenation of nitrobenzene with the CoOₓ@Chit-700 catalyst is constant over five runs. Moreover, also the selectivity of the hydrogenation of nitrobenzene with the CoOₓ@Chit-700 catalyst is constant over three runs.

### Reductive Dehalogenation Processes

Reductive dehalogenation processes of C—X bonds, wherein X is Cl, Br or I, such as processes for dehalogenation of organohalides or processes for deuterium labelling of arenes via dehalogenation of organohalides have many applications in the chemical and pharmaceutical industry.

For example, organohalides, have wide-ranging applications including use in adhesives, aerosols, various solvents, pharmaceuticals, pesticides and fire retardants and as reaction media. However, many organohalides can be toxic to human health and the environment at relatively low concentrations. In view of this potential toxicity, the use and environmentally acceptable emissions of many organohalides is becoming more stringently regulated in Europe and in the Unites States and in many other industrially developed communities. Accordingly, there have been efforts to reduce or eliminate the organohalides, for example pesticides or fire retardants by catalytically converting organohalides to less toxic or nontoxic compounds that have a reduced risk to health and the environment.

Moreover, hydrodehalogenation of organohalides can be used for deuterium labeling of arenes via dehalogenation.

Therefore, in one aspect, the present invention relates to a method for preparing an arene, comprising the step of contacting an organohalide with hydrogen gas in the presence of a nitrogen containing biopolymer-based catalyst, preferably a chitosan-based catalyst of the invention as defined herein. If appropriate the hydrodehalogenation may be carried out in the presence of a suitable base and in the presence of a suitable solvent.

Schemes 5, 6 and 7 illustrate the yields of the corresponding hydrodehalogenated products of substituted organohalides when reacting the substituted organohalides with a nitrogen containing biopolymer-based catalyst, preferably a chitosan-based catalyst of the invention, e.g. with the Co—Co₃Co₄@Chit-700 catalyst. Schemes 5 and 6 summarize the results of the hydrodehalogenation of substituted organohalides in the presence of hydrogen gas, the Co—Co₃Co₄@Chit-700 catalyst and triethylamine in a mixture of methanol and water.

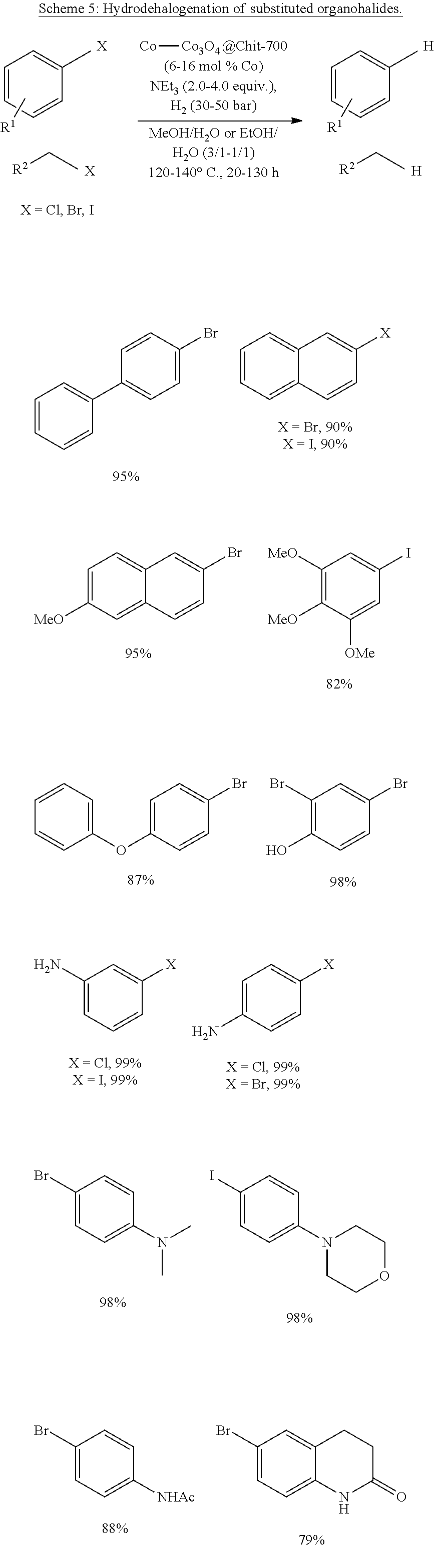

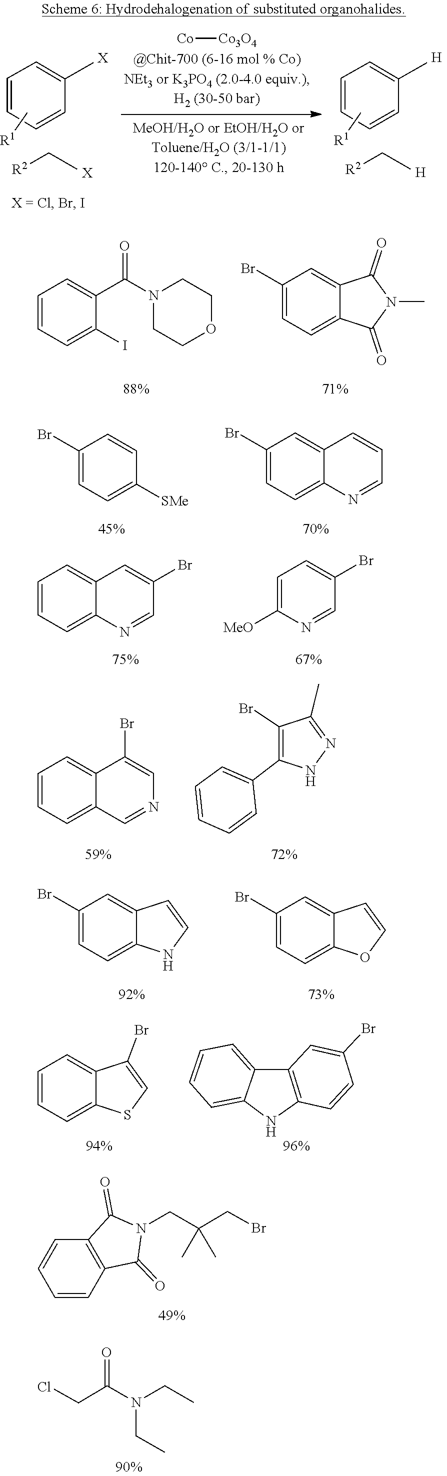

Scheme 7 illustrates the hydrodehalogenation of polysubstituted organohalides in the presence of hydrogen gas, the Co—Co₃Co₄@Chit-700 catalyst of the invention and triethylamine in a mixture of methanol and water. The results show that the Co—Co₃Co₄@Chit-700 catalyst of the invention is suitable for selectively hydrodehalogenating the bromine substituent in polysubstituted organohalides having bromine and chlorine substituents, or bromine and fluorine substituents respectively.

Scheme 7 illustrates the hydrodehalogenation of polysubstituted organohalides.

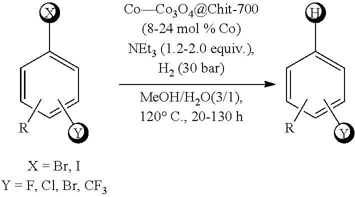

| Entry | Substrate | Product | Yield (%) |
|---|---|---|---|
| 1 | 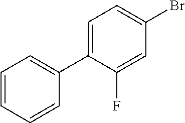 [SMILES: Fc1cc(Br)ccc1-c1ccccc1] | 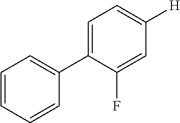 [SMILES: Fc1ccccc1-c1ccccc1] | 93% |
| 2 | 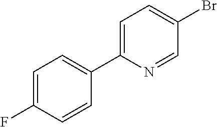 [SMILES: Fc1ccc(-c2ccc(Br)cn2)cc1] | 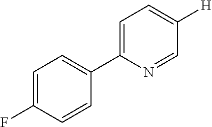 [SMILES: Fc1ccc(-c2ccccn2)cc1] | 90% |
| 3 | 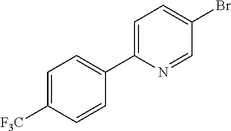 [SMILES: FC(F)(F)c1ccc(-c2ccc(Br)cn2)cc1] | 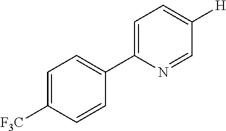 [SMILES: FC(F)(F)c1ccc(-c2ccccn2)cc1] | 88% |
| 4 | 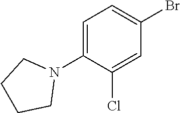 [SMILES: Clc1cc(Br)ccc1N1CCCC1] | 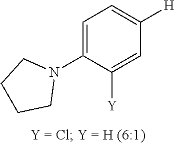 | 91% (overall) |
| 5 | 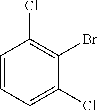 | 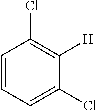 | 73% |
| 6 | 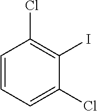 | 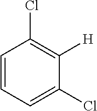 | 87% |
| 7^{c} | 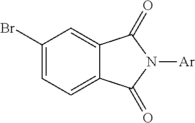 | 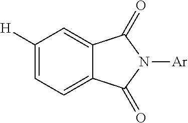 | |
| | 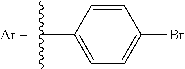 | 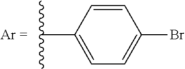 | 46% |

Pesticides or fire retardants may be detoxified by hydrodehalogenation with the nitrogen containing biopolymer-based catalyst, preferably with the chitosan-based catalyst of the invention as defined herein.

Thus, in one aspect, the invention relates to the use of a nitrogen containing biopolymer-based catalyst, preferably a chitosan-based catalyst of the invention as defined herein for detoxifying organohalides, preferably pesticides or fire retardants.

Scheme 8 illustrates detoxification of the pesticides metazachlor and benodanil by hydrodehalogenation with the Co—Co₃Co₄@Chit-700 catalyst of the invention.

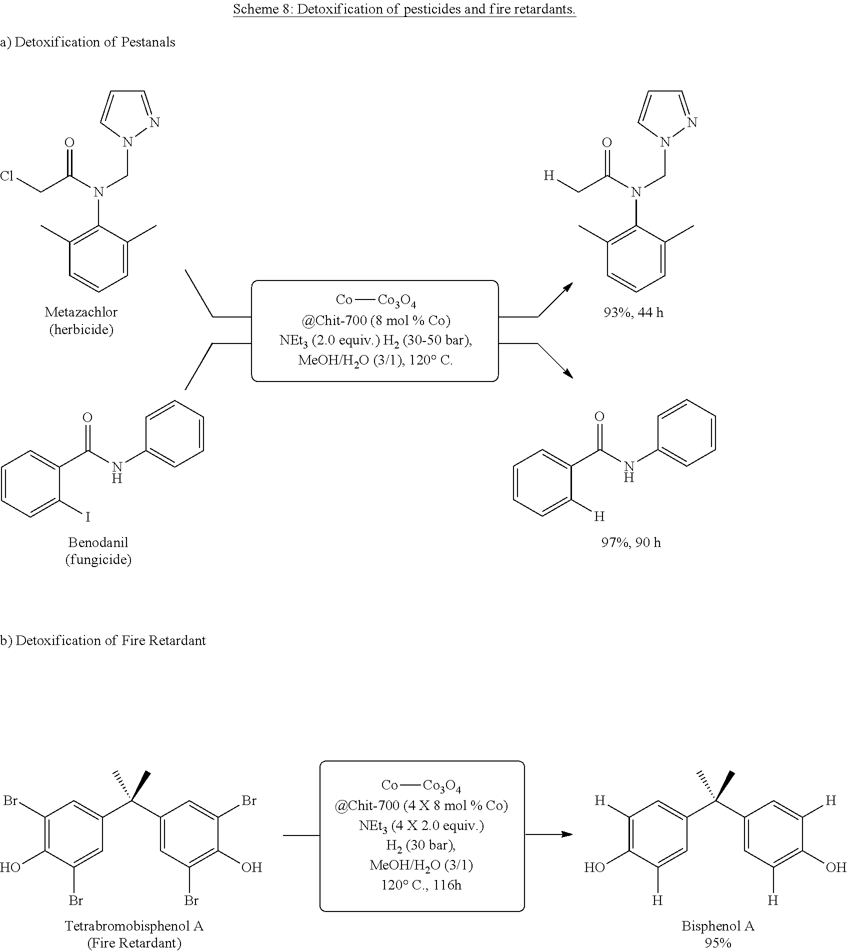

The following examples are provided to aid the understanding of the present invention, the true scope of which is set forth in the appended claims. It is understood that modifications can be made in the procedures set forth without departing from the spirit of the invention.

All patents and publications identified herein are incorporated herein by reference in their entirety.

### EXAMPLES

High resolution scanning transmission electron microscopy (STEM), X-ray diffraction (XRD) and X-ray photoelectron spectroscopy (XPS) were carried out with standard measuring devices.

### Example 1: Preparation of Chitosan-Based Catalysts

### General Procedure for the Preparation of Chitosan-Based Catalysts

Commercially available metal acetate salt was dissolved in absolute ethanol. Then, commercially available chitosan, preferably shrimp shell derived chitosan with low viscosity was added, and the so-obtained suspension was stirred at 70° C. to obtain a metal chitosan complex. Subsequently, the solvent was removed by slow rotary evaporation and the solid metal chitosan complex was dried at 60° C. under vacuum to yield a dried metal chitosan complex. Finally, the dried metal chitosan complex was transferred into a crucible equipped with a lid and pyrolysed at temperatures ranging from 500° C. to 900° C. under an Ar atmosphere to obtain the chitosan-based catalyst of the invention.

### Example 1.1: Preparation of Co—Co₃O₄@Chit-900

Co(OAc)₂.4H₂O+Chitosan→Co/Chitosan→Co—Co₃O₄@Chit-800

126.8 mg (0.5 mmol) of Co(OAc)₂.4H₂O were dissolved in 20 mL of absolute EtOH. Then, 690 mg of chitosan were added and the so-obtained suspension was stirred at 70° C. for 20 h. Subsequently, the solvent was removed by slow rotary evaporation and the solid was dried for 12 h at 60° C. under vacuum. Finally, the dried material was transferred into a crucible equipped with a lid and pyrolysed at 900° C. for 2 h under an Ar atmosphere obtaining the catalytically active material.

### Example 1.2: Preparation of Co—Co₃O₄@Chit-800

Co(OAc)₂.4H₂O+Chitosan→Co/Chitosan→Co—Co₃O₄@Chit-800

126.8 mg (0.5 mmol) of Co(OAc)₂.4H₂O were dissolved in 20 mL of absolute EtOH. Then, 690 mg of chitosan were added and the so-obtained suspension was stirred at 70° C. for 20 h. Subsequently, the solvent was removed by slow rotary evaporation and the solid was dried for 12 h at 60° C. under vacuum. Finally, the dried material was transferred into a crucible equipped with a lid and pyrolysed at 800° C. for 2 h under an Ar atmosphere obtaining the catalytically active material.

### Example 1.3: Preparation of Co—Co₃O₄@Chit-700

Co(OAc)₂.4H₂O+Chitosan→Co/Chitosan→Co—Co₃O₄@Chit-700

126.8 mg (0.5 mmol) of Co(OAc)₂.4H₂O were dissolved in 20 mL of absolute EtOH. Then, 690 mg of chitosan were added and the so-obtained suspension was stirred at 70° C. for 20 h. Subsequently, the solvent was removed by slow rotary evaporation and the solid was dried for 12 h at 60° C. under vacuum. Finally, the dried material was transferred into a crucible equipped with a lid and pyrolysed at 700° C. for 2 h under an Ar atmosphere obtaining the catalytically active material.

### Example 1.4: Synthesis of Co—Co₃O₄@Chit-600

Co(OAc)₂.4H₂O+Chitosan→Co/Chitosan→Co—Co₃O₄@Chit-600

126.8 mg (0.5 mmol) of Co(OAc)₂.4H₂O were dissolved in 20 mL of absolute EtOH. Then, 690 mg of chitosan were added and the so-obtained suspension was stirred at 70° C. for 20 h. Subsequently, the solvent was removed by slow rotary evaporation and the solid was dried for 12 h at 60° C. under vacuum. Finally, the dried material was transferred into a crucible equipped with a lid and pyrolysed at 600° C. for 2 h under an Ar atmosphere obtaining the catalytically active material.

### Example 1.5: Preparation of Co/RNGr-H800 (Co/Renewable N-Doped Graphene/Graphite-Hydrogen800)

Co(OH)₂+Chitosan→Co/Chitosan→Co/RNGr-H800

46.5 mg (0.5 mmol) of Co(OH)₂ were dissolved in 20 mL of absolute EtOH. Then, 690 mg of chitosan were added and the so-obtained suspension was stirred at 70° C. for 4 h. Subsequently, the solvent was removed by slow rotary evaporation and the solid was dried for 5 h under vacuum. Finally, the latter was transferred into a crucible equipped with a lid and pyrolysed at 800° C. for 2 h under an Ar atmosphere obtaining the catalytically active material.

### Example 1.6: Preparation of Co/RNGr-H600 (Co/Renewable N-Doped Graphene/Graphite-Hydrogen600)

Co(OH)₂+Chitosan→Co/Chitosan→Co/RNGr-H600

46.5 mg (0.5 mmol) of Co(OH)₂ were dissolved in 20 mL of absolute EtOH. Then 690 mg of chitosan were added and the so-obtained suspension was stirred at 70° C. for 4 h. Subsequently, the solvent was removed by slow rotary evaporation and the solid was dried for 5 h under vacuum. Finally, the latter was transferred into a crucible equipped with a lid and pyrolysed at 600° C. for 2 h under Ar atmosphere obtaining the catalytically active material.

### Example 1.7: Preparation of Co/RNGr-N800 (Co/Renewable N-Doped Graphene/Graphite-Nitrogen800)

Co(NO₃)₂+Chitosan→Co/Chitosan→Co/RNGr-N800

91.5 mg (0.5 mmol) of Co(NO₃)₂ were dissolved in 20 mL of absolute EtOH. Then, 690 mg of chitosan were added and the so-obtained suspension was stirred at 70° C. for 4 h. Subsequently, the solvent was removed by slow rotary evaporation and the solid was dried for 5 h under vacuum. Finally, the latter was transferred into a crucible equipped with a lid and pyrolysed at 800° C. for 2 h under an Ar atmosphere obtaining the catalytically active material.

### Example 1.8: Preparation of Co/RNGr-N600 (Co/Renewable N-Doped Graphene/Graphite-Nitrogen600)

Co(NO₃)₂+Chitosan→Co/Chitosan→Co/RNGr-N600

91.5 mg (0.5 mmol) of Co(NO₃)₂ were dissolved in 20 mL of absolute EtOH. Then, 690 mg of chitosan were added and the so-obtained suspension was stirred at 70° C. for 4 h. Subsequently, the solvent was removed by slowly rotary evaporation and the solid was dried for 5 h under vacuum. Finally, the latter was transferred into a crucible equipped with a lid and pyrolysed at 600° C. for 2 h under Ar atmosphere obtaining the catalytically active material.

### Example 1.9: Preparation of Cu/RNGr-AC800 (Cu/Renewable N-Doped Graphene/Graphite-Acetate800)

Cu(acac)₂+Chitosan→Cu/Chitosan→Cu/RNGr-AC800

130.9 mg (0.5 mmol) of Cu(acac)₂ were dissolved in 20 mL of absolute EtOH. Then, 690 mg of chitosan were added and the so-obtained suspension stirred at 70° C. for 4 h. Subsequently, the solvent was removed by slow rotary evaporation and the solid as dried for 5 h under vacuum. Finally, the latter was transferred into a crucible equipped with a lid and pyrolysed at 600° C. for 2 h under Ar atmosphere obtaining the catalytically active material.

### Example 1.10: Preparation of Fe/RNGr-A800 (Fe/Renewable N-Doped Graphene/Graphite-Acetate800)

Fe(OAc)₂+Chitosan→Fe/Chitosan→Fe/RNGr-A800

87.0 mg (0.5 mmol) of Fe(OAc)₂ were dissolved in 20 mL of absolute EtOH. Then, 690 mg of chitosan were added and the so-obtained suspension was stirred at 70° C. for 4 h. Subsequently, the solvent was removed by slowly rotary evaporation and the solid was dried for 5 h under vacuum. Finally, the latter was transferred into a crucible equipped with a lid and pyrolysed at 800° C. for 2 h under an Ar atmosphere obtaining the catalytically active material.

### Example 1.11: Preparation of Au/RNGr-C800 (Au/Renewable N-Doped Graphene/Graphite-Carbon800)

HAuCl₄+Chitosan→Au/Chitosan→Au/RNGr-C800

169.9 mg (0.5 mmol) of HAuCl₄ were dissolved in 20 mL of absolute EtOH. Then, 690 mg of chitosan were added and the so-obtained suspension was stirred at 70° C. for 4 h. Subsequently, the solvent was removed by slow rotary evaporation and the solid was dried for 5 h under vacuum. Finally, the latter was transferred into a crucible equipped with a lid and pyrolysed at 800° C. for 2 h under Ar atmosphere obtaining the catalytically active material.

### Example 1.12: Preparation of Ni/RNGr-A800 (Ni/Renewable N-Doped Graphene/Graphite-Acetate800)

Ni(OAc)₂4H₂O+Chitosan→Ni/Chitosan→Ni/RNGr-A800

124.4 mg (0.5 mmol) of Ni(OAc)₂.4H₂O were dissolved in 20 mL of absolute EtOH. Then, 690 mg of chitosan were added and the so-obtained suspension was stirred at 70° C. for 4 h. Subsequently, the solvent was removed by slow rotary evaporation and the solid was dried for 5 h under vacuum. Finally, the latter was transferred into a crucible equipped with a lid and pyrolysed at 800° C. for 2 h under an Ar atmosphere obtaining the catalytically active material.

### Example 1.13: Preparation of Mn/RNGr-C800 (Au/Renewable N-Doped Graphene/Graphite-Carbon800)

MnCl₂+Chitosan→Mn/Chitosan→Mn/RNGr-C800

63.0 mg (0.5 mmol) of MnCl₂ were dissolved in 20 mL of absolute EtOH. Then, 690 mg of chitosan were added and the so-obtained suspension was stirred at 70° C. for 4 h. Subsequently, the solvent was removed by slow rotary evaporation and the solid was dried for 5 h under vacuum. Finally, the latter was transferred into a crucible equipped with a lid and pyrolysed at 800° C. for 2 h under Ar atmosphere obtaining the catalytically active material.

### Example 2: Characterisation of the Chitosan-Based Catalysts

### Example 2.1: Characterisation of the CoOₓ@Chit Catalysts

The CoOₓ@Chit-600 catalyst, the CoOₓ@Chit-700 catalyst, the CoOₓ@Chit-800 catalyst and the CoOₓ@Chit-900 catalyst, which have been prepared from cobalt(II) acetate and shrimp shell-derived chitosan with low viscosity after pyrolysis at 600° C., 700° C., 800° C. and 900° C. respectively, according to Examples 1.4, 1.3, 1.2 and 1.1, respectively, were characterized by elemental analysis. The CoOₓ@Chit-700 catalyst of Example 1.3 was further characterized by means of various analytical techniques, such as high resolution scanning transmission electron microscopy (STEM), X-ray diffraction (XRD), and X-ray photoelectron spectroscopy (XPS).

### Example 2.1.1: Elemental Analysis

The chemical composition of the CoOₓ@Chit-600 catalyst, the CoOₓ@Chit-700 catalyst, the CoOₓ@Chit-800 catalyst and the CoOₓ@Chit-900 catalyst, respectively, was determined by elemental analysis. Table 2 shows that the CoOₓ@Chit-600 catalyst, the CoOₓ@Chit-700 catalyst, the CoOₓ@Chit-800 catalyst and CoOₓ@Chit-900 catalyst respectively, contain the following elements: carbon, hydrogen, nitrogen and cobalt.

Table 2 summarizes the carbon, hydrogen, nitrogen and cobalt content of the catalytic active materials of Examples 1.1, 1.2, 1.3 and 1.4. Table 2 further demonstrates that with the increase of the pyrolysis temperature (600° C. to 900° C.) in the carbonization process, the content of carbon in the catalyst increases. In contrast thereto, the content of nitrogen in the catalyst decreases with the increase of the pyrolysis temperature (600° C. to 900° C.) in the carbonization process.

TABLE 2
Elemental analysis of the pyrolysed materials

| | Example | C | H | N | Co |
|---|---|---|---|---|---|
| Catalyst | no. | (wt %) | (wt %) | (wt %) | (wt %) |
| CoOₓ@Chit-600 | 1.4 | 70.16 | 1.14 | 6.65 | 8.44 |
| CoOₓ@Chit-700 | 1.3 | 73.78 | 0.60 | 3.23 | 9.76 |
| CoOₓ@Chit-800 | 1.2 | 78.81 | 0.69 | 3.19 | 9.32 |
| CoOₓ@Chit-900 | 1.1 | 79.10 | 0.15 | 3.09 | 10.49 |

### Example 2.1.2: Characterization of the CoOₓ@Chit-700 Catalyst by Scanning Transmission Electron Microscopy (STEM), X-Ray Diffraction (XRD) and X-Ray Photoelectron Spectroscopy (XPS)

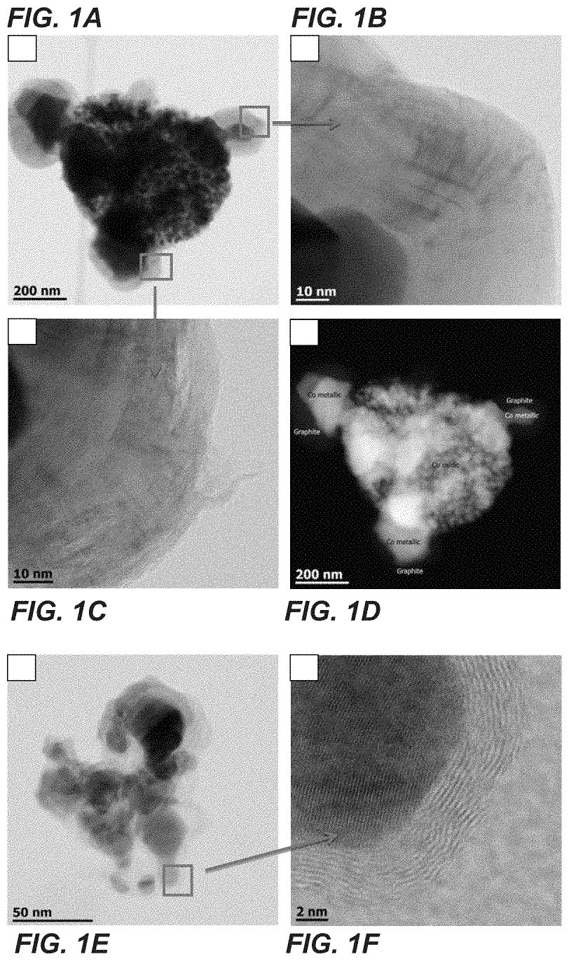

In order to obtain structural insight, the CoOₓ@Chit-700 catalyst was characterized by STEM measurements. FIG. 1 shows high resolution scanning transmission electron microscopy (STEM) images of the CoOₓ@Chit-700 catalyst. FIGS. 1A, 1B, 1C, 1E, and 1F show annular bright field (ABF) images of the CoOₓ@Chit-700 catalyst. FIG. 1D shows high-angle annular dark field (HAADF) images of cobalt composites of the catalyst. High-angle annular dark field (HAADF) measurements were carried out with the help of spherical aberration (Cs)-corrected scanning transmission electron microscope (STEM).

FIGS. 1B and 1C are cutouts of FIG. 1A, and show annular bright field (ABF) images of the CoOₓ@Chit-700 catalyst. The images demonstrate that metallic cobalt particles are embedded in graphitic shells of more than 50 nm thickness.

FIGS. 1E and 1F are also STEM images of the CoOₓ@Chit-700 catalyst.

FIGS. 1A, 1C, 1E, and 1F show that the thickness of the graphitic layers varies from region to region. In some regions, there are more than 140 layers (FIGS. 1A and 1C), while other regions have only 10 layers (FIGS. 1E and 1F).

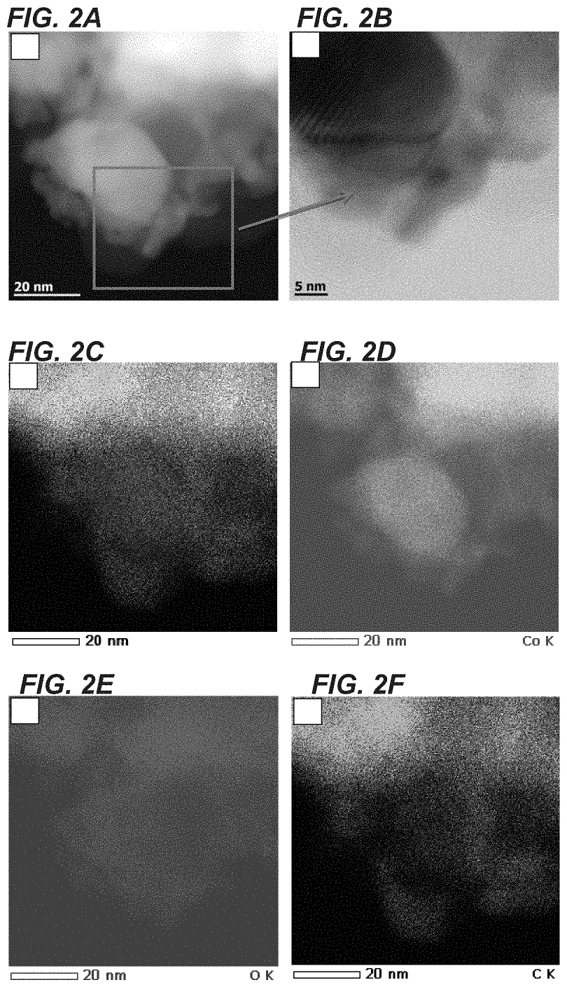

FIGS. 2A, 2C, 2D, 2E, and 2F show energy-dispersive X-ray spectroscopy (EDXS) images and mapping of the CoOₓ@Chit-700 catalyst. FIGS. 2A, 2C, 2D, 2E, and 2F demonstrate best partially oxidized cobalt phase, where metallic cobalt core is partially enveloped by cobalt oxide crystallites and embedded in the graphitic carbon matrix. Mostly, thin graphite layers were observed (FIGS. 2A and 2B) as shown also in ABF images (FIGS. 1A, 1C, 1E, and 1F). All the observed cobalt structures, partially oxidized and completely metallic cobalt, can exist in different states due to the Kirkendall effect on Co nanoparticles as described by H. J. Fan et al. (H. J. Fan et al, Small 2007, 3, 16660-1671), G. E. Murch et al. (E. Murch et al., diffusion-fundamentals.org 2009, 11, 1-22) and C.-M. Wang et al. (C.-M. Wang et al., Sci. Rep. 2014, 4, 3683).

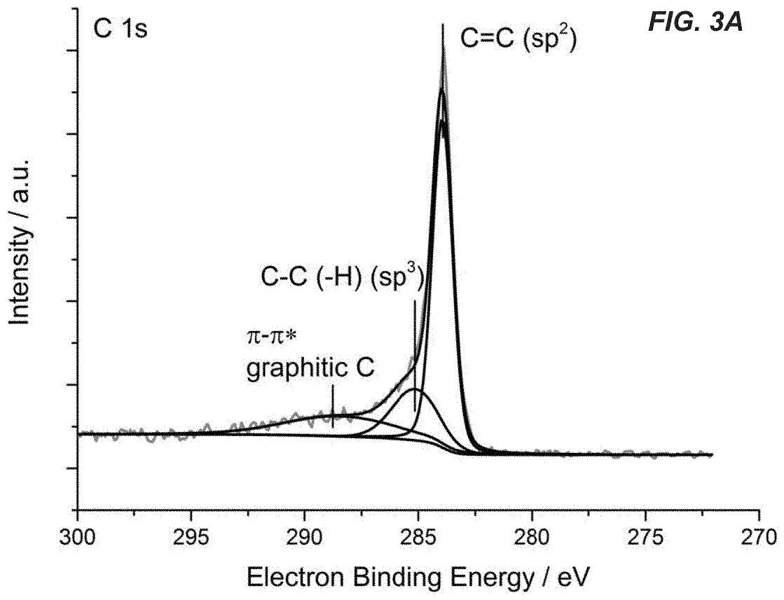

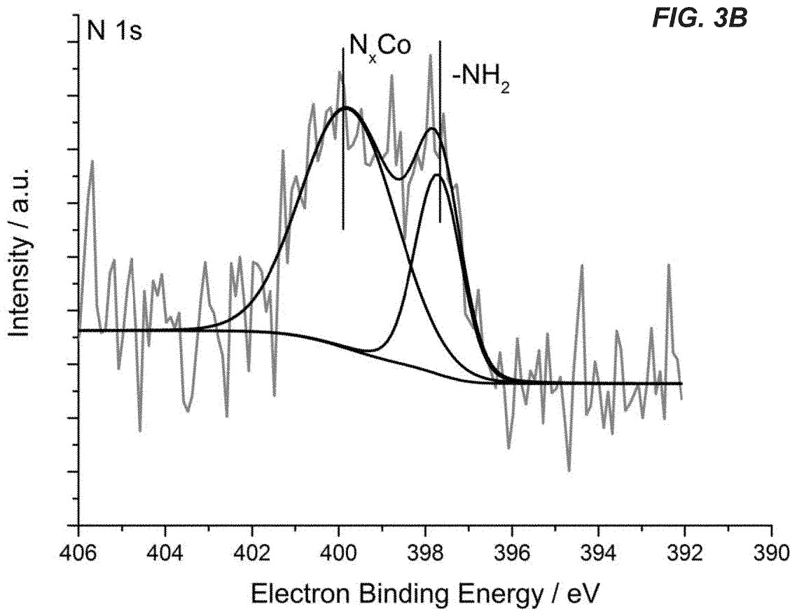

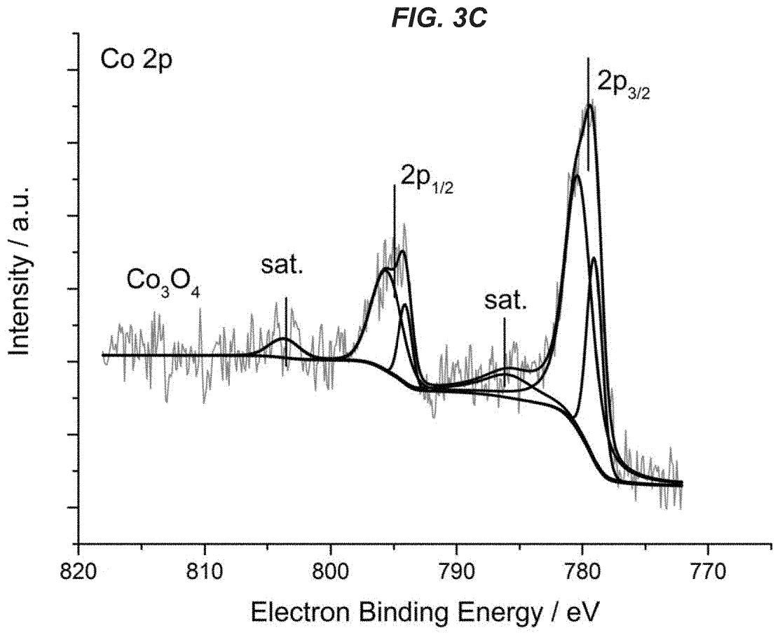

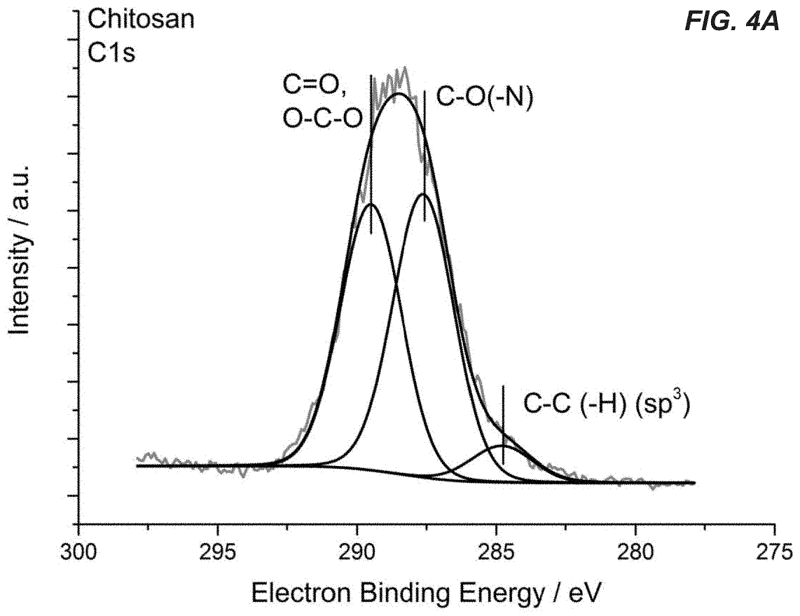

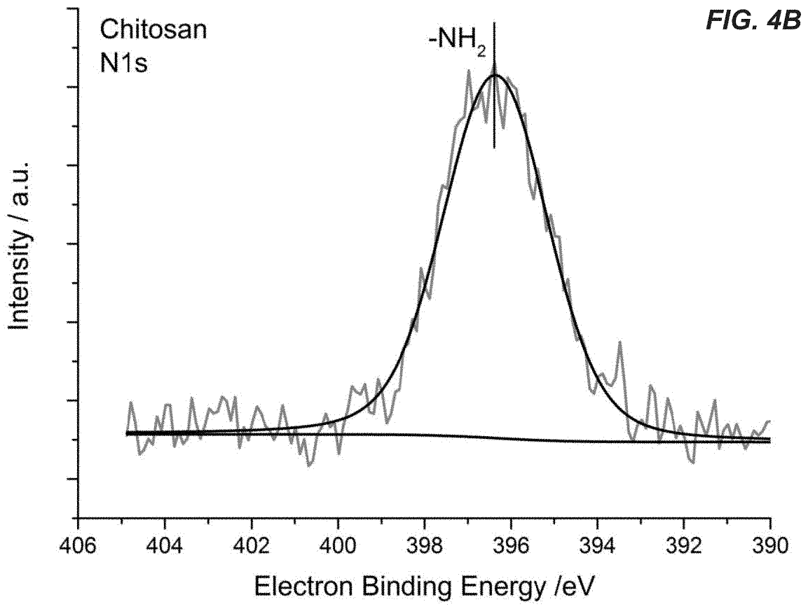

In order to further investigate the composition of the CoOₓ@Chit-700 catalyst, X-ray photoelectron spectroscopy (XPS) measurements were carried out, which reveal the presence of carbon, nitrogen, oxygen and cobalt in the regions including surface and few layers underneath the surface of the catalyst. FIGS. 3A-3D are XPS spectra of the CoOₓ@Chit-700 catalyst. Furthermore, XPS comparison spectra of pure chitosan were recorded and are shown in FIGS. 4A and 4B.

As shown in FIG. 3A, the C1s spectrum of this catalyst consists of three different peaks: C(sp²) (C═C), C(sp³) (C—C or C—H) and graphitic C with corresponding electron-binding energy of 283.9, 285.1, 288.4 eV. C(sp²) (C═C) and graphitic carbon are obtained in the carbonization process, while C(sp³) (C—C or C—H) most probably results from unpyrolysed chitosan (FIG. 4A).

The N1s spectrum clearly displays at least two different peaks: the lower binding energy peak was observed in unpyrolysed chitosan, too, and correlated to the amine nitrogen (NH₂) (FIG. 4B); The higher binding energy peak can be explained by the bonding to the cobalt ions (FIG. 3B). The measured Co2p spectrum, shows the presence of only Co₃O₄ species on the surface and few layers underneath of the cobalt composites (FIG. 3C). Further, the spectrum corresponds to the Co₃O₄ data reported by M. C. Biesinger et al., Appl. Surf. Sci. 2011, 257, 2717-2730.

The contents of C, N, O and Co calculated by XPS analysis are 73.83%, 2.06%, 13.74% and 10.37% respectively (all in weight %). The slight changes in the nitrogen and cobalt contents of this catalyst can be attributed to the analytic differences, since elemental analysis is involved in the measurement of whole material while XPS analysis measures for the surface and few layers underneath.

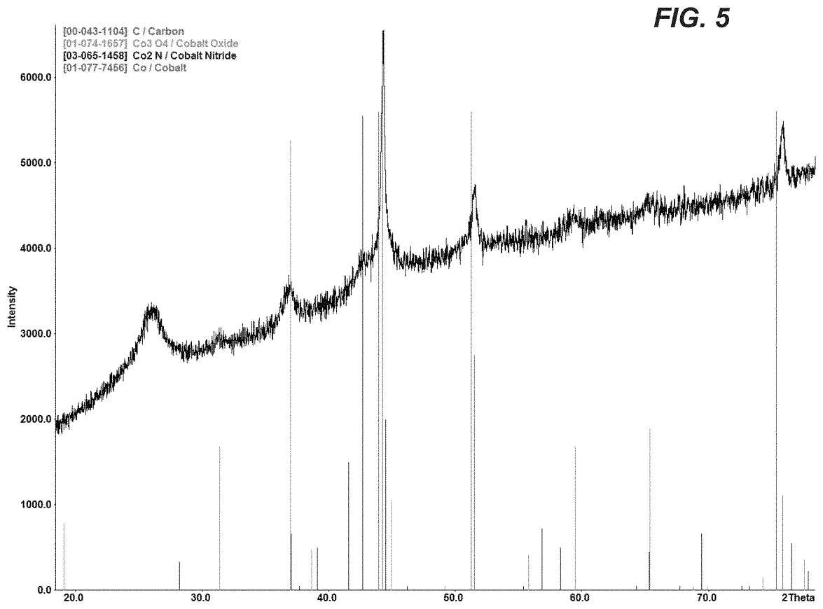

In order to obtain more insight into the composition of cobalt composites, X-ray diffraction (XRD) measurements were also carried out. The XRD spectrum of the CoOₓ@Chit-700 catalyst is shown in FIG. 5. In the XRD spectrum, the strong signals for the reflections from metallic cobalt (2θ=44.23°, 51.53° and 75.87°) and oxidic cobalt (Co₃O₄) (2θ=19.04°, 31.35°, 36.94°, 38.64°, 44.92°, 55.80°, 59.51°, 65.41°, 74.32° and 77.56°) were observed. These observations are in agreement with the HAADF and XPS results. In addition, weak signals for the reflections probably from cobalt nitrogen containing species (2θ=37.03°, 39.08°, 41.54°, 42.66°, 44.49°, 56.85°, 58.35°, 65.35°, 69.47° and 76.56°) were also observed.

### Summary of the Characterization by STEM, XRD and XPS

Based on the analytical results, the CoOₓ@Chit-700 catalyst is composed of metallic cobalt partially enveloped with cobalt oxide shell embedded in the graphitic carbon matrix and can be designated as Co—Co₃O₄@Chit-700.

### Example 3: Hydrogenation of Nitroarenes

### Example 3.1: Preparation of Substituted Anilines from Nitroarenes

### Example 3.1.1: General Procedure for the Preparation of Substituted Anilines from Nitroarenes

In a 4 mL reaction glass vial fitted with a septum cap containing a magnetic stirring bar, Co—Co₃O₄@Chit-700 (10 mg, 3.4 mol % Co), the nitroarenes (0.5 mmol, 1.0 equiv.) and triethylamine (35 μL, 0.25 mmol, 0.5 equiv.) were added to a solvent mixture of EtOH/H₂O (3/1, 2 mL). The reaction vial was then placed into a 300 mL autoclave, flashed with hydrogen five times and finally pressurized to 40 bar. The reaction mixture was stirred for appropriate time at 110° C. After cooling the reaction mixture to room temperature, the autoclave was slowly depressurized. The crude reaction mixture was filtered through a pipette fitted with a cotton bed and the solvent was evaporated under reduced pressure. The crude products were purified by passing through a silica plug (eluent: ethyl acetate) to give pure aniline derivatives after removal of solvent.

The following compounds may be prepared from the respective nitroarenes using the catalyst of the invention:

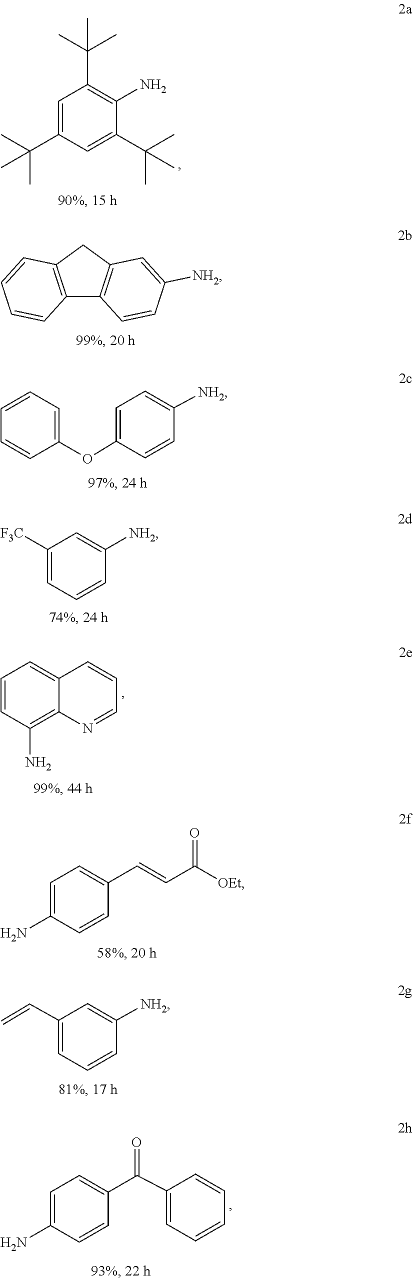

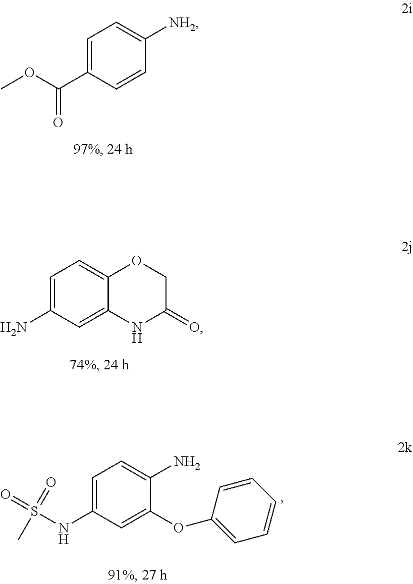 [SMILES: COC(=O)c1ccc(N)cc1 ; Nc1ccc2c(c1)NC(=O)CO2 ; CS(=O)(=O)Nc1ccc(N)c(Oc2ccccc2)c1]

### Example 3.1.2: Preparation of 2,4,6-Tri-tert-butylaniline (2a)

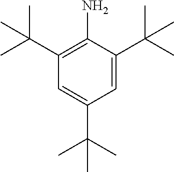 [SMILES: CC(C)(C)c1cc(C(C)(C)C)c(N)c(C(C)(C)C)c1]

Reaction Time: 15 h; Isolated Yield: 90%; ¹H NMR (300 MHz, CDCl₃): δ (ppm): 7.07 (s, 2H), 3.87 (bs, 2H), 1.29 (s, 18H), 1.12 (s, 9H); ¹³C NMR (75 MHz, CDCl₃): δ (ppm): 141.1, 139.3, 133.6, 122.0, 34.9, 34.6, 31.9, 30.5.

### Example 3.1.3: Preparation of 9H-Fluoren-2-amine (2b)

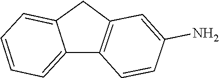 [SMILES: Nc1ccc2c(c1)Cc1ccccc1-2]

Reaction Time: 20 h; Isolated Yield: 99%; ¹H NMR (400 MHz, CDCl₃): δ (ppm): 7.65 (dt, J=7.5, 0.9 Hz, 1H), 7.58 (d, J=8.1 Hz, 1H), 7.48 (dt, J=7.5, 1.0 Hz, 1H), 7.33 (tt, J=7.5, 0.9 Hz, 1H), 7.21 (td, J=7.4, 1.1 Hz, 1H), 6.88 (dd, J=2.0, 0.9 Hz, 1H), 6.72 (dd, J=8.1, 2.2 Hz, 1H), 3.82 (s, 2H), 3.74 (bs, 2H); ¹³C NMR (101 MHz, CDCl₃): δ (ppm): 145.9, 145.3, 142.4, 142.3, 133.1, 126.7, 125.2, 124.9, 120.8, 118.7, 114.1, 111.9, 36.9.

### Example 3.1.4: Preparation of 4-phenoxyaniline (2c)

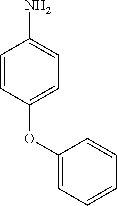 [SMILES: Nc1ccc(Oc2ccccc2)cc1]

Reaction Time: 24 h; Isolated Yield: 97%; ¹H NMR (300 MHz, CDCl₃): δ (ppm): 7.23-7.35 (m, 2H), 7.02 (t, J=7.3 Hz, 1H), 6.94 (d, J=8.0 Hz, 2H), 6.88 (d, J=8.6 Hz, 2H), 6.68 (d, J=8.6 Hz, 2H), 3.57 (bs, 2H); ¹³C NMR (75 MHz, CDCl₃): δ (ppm): 159.0, 148.7, 142.8, 129.6, 122.2, 121.3, 117.4, 116.4.

### Example 3.1.5: Preparation of 3-(trifluoromethyl)aniline (2d)

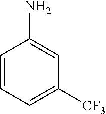 [SMILES: Nc1cccc(C(F)(F)F)c1]

Reaction Time: 24 h; Isolated Yield: 74%; ¹H NMR (300 MHz, CDCl₃): δ (ppm): 7.31-7.36 (m, 1H), 7.08 (d, J=7.7 Hz, 1H), 6.98 (s, 1H), 6.90 (dd, J=8.1, 2.4 Hz, 1H), 3.91 (bs, 2H); ¹³C NMR (75 MHz, CDCl₃): δ (ppm): 146.8, 131.7 (q, J=31.8 Hz), 129.9, 124.3 (q, J=272.3 Hz), 118.1, 115.1 (q, J=4.1 Hz), 111.4 (q, J=3.9 Hz); ¹⁹F NMR (300 MHz, CDCl₃): δ (ppm): −62.49.

### Example 3.1.6: Preparation of quinolin-8-amine (2e)

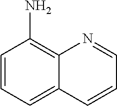 [SMILES: Nc1cccc2cccnc12]

Reaction Time: 44 h; Isolated Yield: 99%; ¹H NMR (300 MHz, CDCl₃): δ (ppm): 8.69 (dd, J=4.1, 1.8 Hz, 1H), 7.97 (dd, J=8.3, 1.8 Hz, 1H), 7.23-7.29 (m, 2H), 7.07 (dd, J=8.3, 1.3 Hz, 1H), 6.85 (dd, J=7.5, 1.3 Hz, 1H), 4.95 (bs, 2H); ¹³C NMR (75 MHz, CDCl₃): δ (ppm): 147.5, 144.1, 138.5, 136.0, 128.9, 127.4, 121.4, 116.0, 110.1.

### Example 3.1.7: Preparation of ethyl (E)-3-(4-aminophenyl)acrylate (2f)

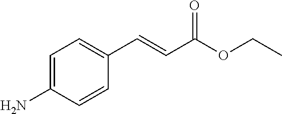

Reaction Time: 20 h; Isolated Yield: 58%; ¹H NMR (300 MHz, CDCl₃): δ (ppm): 7.59 (d, J=15.9 Hz, 1H), 7.34 (d, J=8.0 Hz, 2H), 6.64 (d, J=8.5 Hz, 2H), 6.23 (d, J=15.9 Hz, 1H), 4.24 (q, J=7.1 Hz, 2H), 3.95 (bs, 2H), 1.32 (t, J=7.1 Hz, 3H); ¹³C NMR (75 MHz, CDCl₃): δ (ppm): 167.8, 148.8, 145.0, 130.0, 124.9, 114.9, 113.9, 60.3, 14.5.

### Example 3.1.8: Preparation of 3-vinylaniline (2g)

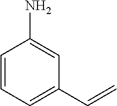

Reaction Time: 17 h; Isolated Yield: 81%; ¹H NMR (300 MHz, CDCl₃): δ (ppm): 7.13 (t, J=7.8 Hz, 1H), 6.84 (d, J=7.6 Hz, 1H), 6.57-6.74 (m, 3H), 5.71 (dd, J=17.5, 1.0 Hz, 1H), 5.22 (dd, J=10.9, 1.0 Hz, 1H), 3.60 (bs, 2H); ¹³C NMR (75 MHz, CDCl₃): δ (PPM): 146.6, 138.7, 137.1, 129.5, 117.0, 114.9, 113.7, 112.8.

### Example 3.1.9: Preparation of (4-aminophenyl)(phenyl)methanone (2h)

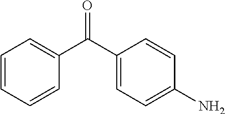 [SMILES: Nc1ccc(C(=O)c2ccccc2)cc1]

Reaction Time: 22 h; GC Yield: 93% (determined by GC-FID analysis using hexadecane as internal standard).

### Example 3.1.10: Preparation of methyl 4-aminobenzoate (2i)

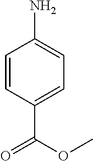

Reaction Time: 24 h; Isolated Yield: 97%; ¹H NMR (300 MHz, CDCl₃): δ (ppm): 7.83 (d, J=8.8 Hz, 2H), 6.61 (d, J=8.8 Hz, 2H), 4.22 (bs, 2H), 3.83 (s, 3H); ¹³C NMR (75 MHz, CDCl₃): δ (PPM): 167.3, 151.1, 131.6, 119.3, 113.8, 51.6.

### Example 3.1.11: 6-amino-2H-benzo[b][1,4]oxazin-3(4H)-one (2j)

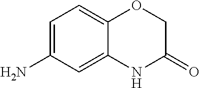 [SMILES: Nc1ccc2c(c1)NC(=O)CO2]

Reaction Time: 24 h; Isolated Yield: 74%; ¹H NMR (300 MHz, DMSO-d₆): δ (ppm): 10.44 (s, 1H), 6.61 (d, J=8.4 Hz, 1H), 6.17 (d, J=2.6 Hz, 1H), 6.12 (dd, J=8.4, 2.6 Hz, 1H), 4.84 (bs, 2H), 4.36 (s, 2H); ¹³C NMR (75 MHz, DMSO-d₆): δ (ppm): 165.7, 144.1, 134.2, 127.6, 116.3, 108.3, 101.5, 67.0.

### Example 3.1.12: N-(4-amino-3-phenoxyphenyl)methanesulfonamide (2k)

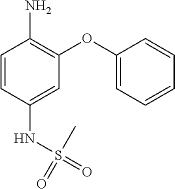 [SMILES: CS(=O)(=O)Nc1ccc(N)c(Oc2ccccc2)c1]

Reaction Time: 27 h; Isolated Yield: 91%; ¹H NMR (300 MHz, DMSO-d₆): δ (ppm): 8.77 (bs, 1H), 7.41 (m, 2H), 7.00-7.18 (m, 4H), 6.29-6.32 (m, 1H), 6.06 (s, 1H), 5.26 (bs, 2H), 2.88 (s, 3H); ¹³C NMR (75 MHz, DMSO-d₆): δ (ppm): 156.3, 153.2, 149.2, 130.5, 129.9, 123.6, 119.3, 114.9, 108.9, 102.9, 40.1.

### Example 3.2: Hydrogenation of Nimesulide and Flutamide

The two pharmaceutical drugs nimesulide and flutamide were reacted under standard reaction conditions according to the general procedure to afford the corresponding amine analogues in 91% and 97% yields, respectively and excellent selectivity.

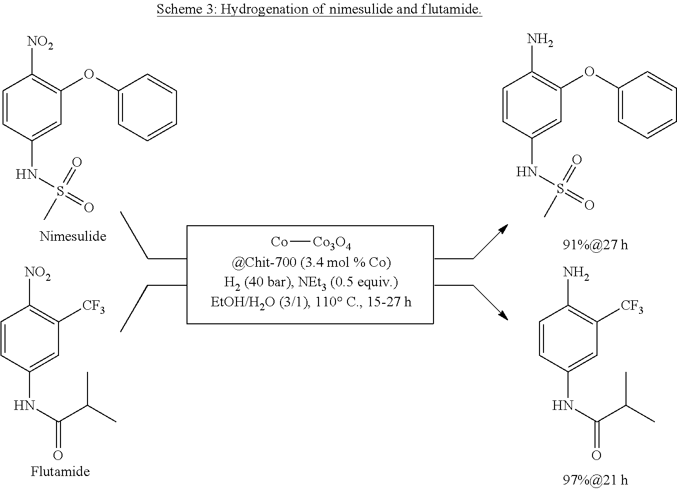

### Example 3.3. Comparison Between CoOx@Chitosan-600/700/800/900 in the Hydrogenation of Nitrobenzene

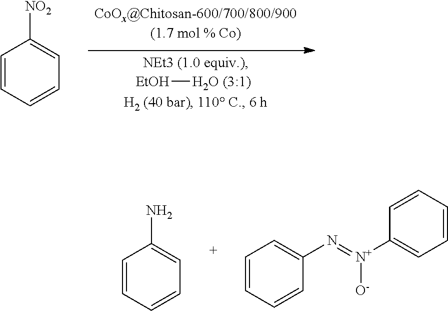

In a 4 mL reaction glass vial fitted with a septum cap containing a magnetic stirring bar, CoOx@Chitosan-600/700/800/900 (4.5-5.5 mg, 1.7 mol % Co), the nitrobenzene (0.5 mmol, 1.0 equiv.) and triethylamine (70 μL, 0.5 mmol, 1.0 equiv.) were added to a solvent mixture of EtOH/H₂O (3/1, 2 mL). The reaction vial was then placed into a 300 mL autoclave, flashed with hydrogen five times and finally pressurized to 40 bar. The reaction mixture was stirred for appropriate time at 110° C. After cooling the reaction mixture to room temperature, the autoclave was slowly depressurized. The crude reaction mixture was filtered through a pipette fitted with a cotton bed and the solvent was evaporated under reduced pressure. The crude products were purified by passing through a silica plug (eluent: ethyl acetate) to give pure aniline derivatives after removal of solvent.

TABLE 3
Results of CoOx@Chitosan-600/700/800/900
in the Hydrogenation of Nitrobenzene

| Catalyst | | H₂ | T | Time | | Conv | |
|---|---|---|---|---|---|---|---|
| (M-mol %) | Solvent | (bar) | ( C. °) | (h) | Additive | (%) | Selectivity |
| CoOₓ@Chitosan- | EtOH—H₂O | 40 | 110 | 6 | NEt₃ (1) | 14 | >99 |
| 600 (1.7% Co) | (3:1) | | | | | | |
| CoOₓ@Chitosan- | EtOH—H₂O | 40 | 110 | 6 | NEt₃ (1) | 65 | >99 |
| 700 (1.7% Co) | (3:1) | | | | | | |
| CoOₓ@Chitosan- | EtOH—H₂O | 40 | 110 | 6 | NEt₃ (1) | 27 | >99 |
| 800 (1.7% Co) | (3:1) | | | | | | |
| CoOₓ@Chitosan- | EtOH—H₂O | 40 | 110 | 6 | NEt₃ (1) | 49 | 98 |
| 900 (1.7% Co) | (3:1) | | | | | | |

### Example 4: Hydrodehalogenation of Organohalides

### Example 4.1: Preparation of Substituted Arenes from Substituted Organohalides

### Example 4.1.1: General Procedure for the Preparation of Substituted Arenes from Substituted Organohalides

In a 4 mL or 8 mL reaction glass vial fitted with a septum cap containing a magnetic stirring bar, Co—Co₃O₄@Chitosan-700, the halogen containing compounds and NEt₃ or K₃PO₄ were added to a solvent mixture. The reaction vial was then placed into a 300 mL autoclave, flashed with hydrogen five times and finally pressurized to 30-50 bar. The reaction mixture was stirred for appropriate time at 120-140° C. After cooling the reaction mixture to room temperature, the autoclave was slowly depressurized. The crude reaction mixture was filtered through a pipette fitted with a cotton bed and the solvent was evaporated under reduced pressure. The crude products were purified by flash column chromatography (eluent: heptane/ethyl acetate) to give pure products.

### Example 4.2: Detoxification of Pesticides

The two pesticides metazachlor and benodanil were degraded to the corresponding hydrodehalogenated analogues according to the general procedure in very good yields in the presence of catalyst, triethylamine and hydrogen gas.

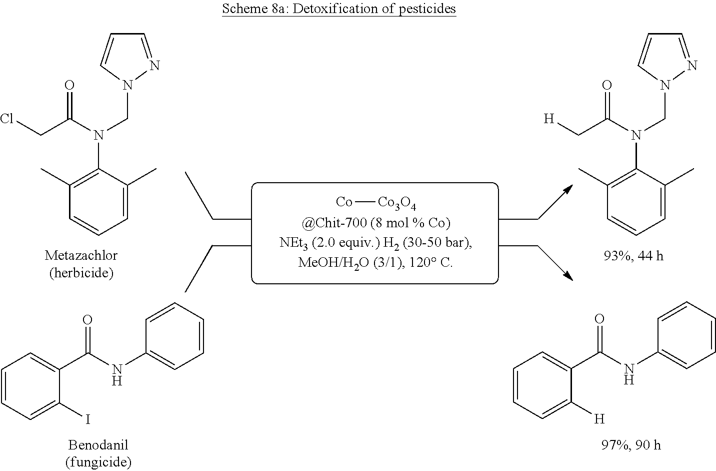

### Example 4.3: Detoxification of Fire Retardants

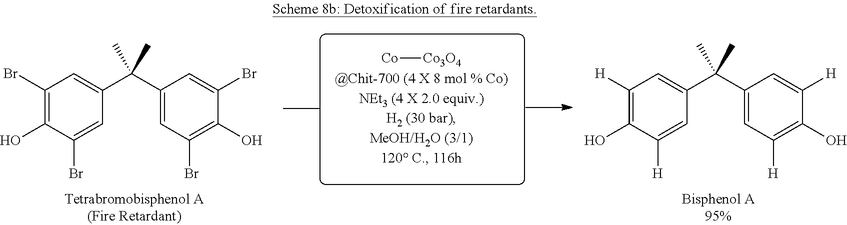

Tetrabromobisphenol A was reacted according to the general procedure with hydrogen gas in the presence of catalyst and trimethylamine at 120° C. to degrade to non-toxic Bisphenol A.

### Example 5: Preparation of Chitin-based Catalysts

### General Procedure for the Preparation of Chitin-Based Catalysts

Commercially available metal acetate salt was dissolved in absolute ethanol. Then, commercially available chitin, preferably shrimp shell derived chitin with practical grade powder was added, and the so-obtained suspension was stirred at 70° C. to obtain a metal chitin complex. Subsequently, the solvent was removed by slow rotary evaporation and the solid metal chitin complex was dried at 60° C. under vacuum to yield a dried metal chitin complex. Finally, the dried metal chitin complex was transferred into a crucible equipped with a lid and pyrolysed at temperatures ranging from 700° C. to 800° C. under an Ar atmosphere to obtain the chitin-based catalyst of the invention.

### Example 5.1: Preparation of MOₓChitin 700/800 Catalysts

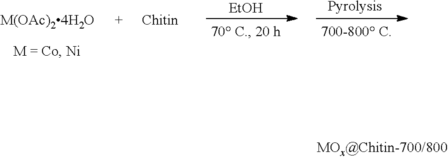

### Example 5.1.1: Preparation of CoOₓChitin 700

126.8 mg (0.5 mmol) of Co(OAc)₂.4H₂O were dissolved in 20 mL of absolute EtOH. Then, 700 mg of chitin were added and the so-obtained suspension was stirred at 70° C. for 20 h. Subsequently, the solvent was removed by slow rotary evaporation and the solid was dried for 12 h at 60° C. under vacuum. Finally, the dried material was transferred into a crucible equipped with a lid and pyrolysed at 700° C. for 2 h under an Ar atmosphere obtaining the catalytically active material.

### Example 5.1.2: Preparation of CoOₓChitin 800

126.8 mg (0.5 mmol) of Co(OAc)₂.4H₂O were dissolved in 20 mL of absolute EtOH. Then, 700 mg of chitin were added and the so-obtained suspension was stirred at 70° C. for 20 h. Subsequently, the solvent was removed by slow rotary evaporation and the solid was dried for 12 h at 60° C. under vacuum. Finally, the dried material was transferred into a crucible equipped with a lid and pyrolysed at 800° C. for 2 h under an Ar atmosphere obtaining the catalytically active material.

### Example 5.1.3: Preparation of NiOₓChitin 700

124.4 mg (0.5 mmol) of Ni(OAc)₂.4H₂O were dissolved in 20 mL of absolute EtOH. Then, 700 mg of chitin were added and the so-obtained suspension was stirred at 70° C. for 20 h. Subsequently, the solvent was removed by slow rotary evaporation and the solid was dried for 12 h at 60° C. under vacuum. Finally, the dried material was transferred into a crucible equipped with a lid and pyrolysed at 700° C. for 2 h under an Ar atmosphere obtaining the catalytically active material.

### Example 5.1.4: Preparation of NiOₓChitin 800

124.4 mg (0.5 mmol) of Ni(OAc)₂.4H₂O were dissolved in 20 mL of absolute EtOH. Then, 700 mg of chitin were added and the so-obtained suspension was stirred at 70° C. for 20 h. Subsequently, the solvent was removed by slow rotary evaporation and the solid was dried for 12 h at 60° C. under vacuum. Finally, the dried material was transferred into a crucible equipped with a lid and pyrolysed at 800° C. for 2 h under an Ar atmosphere obtaining the catalytically active material.

TABLE 4
Elemental Analysis of MOₓChitin 700/800 catalysts (M = Co, Ni)

| Metal Source | Ligand | Pyrolysis (° C.) | C (wt %) | H (wt %) | N (wt %) | M (wt %) |
|---|---|---|---|---|---|---|
| Co(OAc)₂•4H₂O | Chitin | 700 | 70.56 | 0.264 | 2.326 | 11.783 |
| Co(OAc)₂•4H₂O | Chitin | 800 | 74.04 | 0.165 | 2.02 | 11.356 |
| Ni(OAc)₂•4H₂O | Chitin | 700 | 68.69 | 0.495 | 5.052 | 13.381 |
| Ni(OAc)₂•4H₂O | Chitin | 800 | 68.45 | 0.350 | 3.403 | 14.266 |

### Example 6: Hydrogenation of Nitrobenzene with MOₓChitin 700/800 Catalysts (M=Co,Ni)

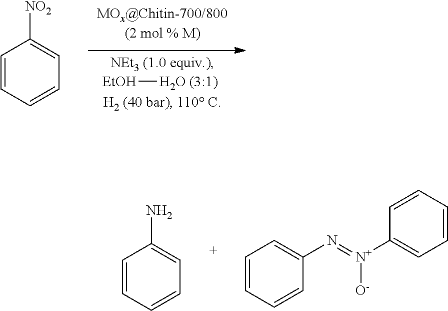

### Example 6.1: General Procedure for the Hydrogenation of Nitrobenzene

In a 4 mL reaction glass vial fitted with a septum cap containing a magnetic stirring bar MOₓChitin 700/800 M=Co,Ni) (4.2-5.2 mg, 2.0 mol % M), the nitroarenes (0.5 mmol, 1.0 equiv.) and triethylamine (70 μL, 0.5 mmol, 1.0 equiv.) were added to a solvent mixture of EtOH/H₂O (3/1, 2 mL). The reaction vial was then placed into a 300 mL autoclave, flashed with hydrogen five times and finally pressurized to 40 bar. The reaction mixture was stirred for appropriate time at 110° C. After cooling the reaction mixture to room temperature, the autoclave was slowly depressurized. The crude reaction mixture was filtered through a pipette fitted with a cotton bed and the solvent was evaporated under reduced pressure. The crude products were purified by passing through a silica plug (eluent: ethyl acetate) to give pure aniline derivatives after removal of solvent.

TABLE 5
Results of the Hydrogenation of Nitrobenzene MOₓChitin
700/800 catalysts (M = Co, Ni)

| Catalyst | | H₂ | | Time | | Conv | |
|---|---|---|---|---|---|---|---|
| (M-mol %) | Solvent | (bar) | T (C. ° C.) | (h) | Additive | (%) | Selectivity |
| CoOx@Chitin- | EtOH—H₂O | 40 | 110 | 2 | NEt₃ | 42 | 97 |
| 700 (2% Co) | (3:1) | | | | | | |
| NiOx@Chitin- | EtOH—H₂O | 40 | 110 | 2 | NEt₃ | 49 | 87 |
| 700 (2% Ni) | (3:1) | | | | | | |
| CoOx@Chitin- | EtOH—H₂O | 40 | 110 | 4 | NEt₃ | 81 | >99 |
| 700 (2% Co) | (3:1) | | | | | | |
| NiOx@Chitin- | EtOH—H₂O | 40 | 110 | 4 | NEt₃ | >99 | >99 |
| 700 (2% Ni) | (3:1) | | | | | | |
| CoOx@Chitin- | EtOH—H₂O | 40 | 110 | 2 | NEt₃ | 43 | 95 |
| 800 (2% Co) | (3:1) | | | | | | |
| NiOx@Chitin- | EtOH—H₂O | 40 | 110 | 2 | NEt₃ | 46 | 79 |
| 800 (2% Ni) | (3:1) | | | | | | |
| CoOx@Chitin- | EtOH—H₂O | 40 | 110 | 4 | NEt₃ | 98 | >99 |
| 800 (2% Co) | (3:1) | | | | | | |
| NiOx@Chitin- | EtOH—H₂O | 40 | 110 | 4 | NEt₃ | >99 | >99 |
| 800 (2% Ni) | (3:1) | | | | | | |

1-28. (canceled)

29. A method of:
hydrogenation of a nitroarene, nitrile, or imine, comprising contacting a nitroarene, a nitrile or an imine with hydrogen gas in the presence of a nitrogen containing biopolymer-based catalyst; or
reductive dehalogenation of an organohalide, comprising contacting an organohalide with hydrogen gas in the presence of a nitrogen containing biopolymer-based catalyst;
wherein the nitrogen containing biopolymer-based catalyst is prepared by a method comprising the steps of:
(a) mixing a metal precursor containing a transition metal, wherein the transition metal is nickel or cobalt, in the presence of an alcohol with a nitrogen containing biopolymer, to obtain a metal complex with the nitrogen containing biopolymer; wherein the nitrogen containing biopolymer is chitosan or chitin;
(b) drying under vacuum the metal complex with the nitrogen containing biopolymer; and
(c) pyrolysing the metal complex with the nitrogen containing biopolymer at temperatures ranging from 550° C. to 850° C. in an inert gas atmosphere to obtain the nitrogen containing biopolymer-based catalyst.

30. The method of claim 29, wherein the transition metal is cobalt.

31. The method of claim 29, wherein the alcohol is ethanol.

32. The method of claim 29, wherein the metal precursor is a metal salt selected from the group consisting of acetate, bromide, chloride, iodide, hydrochloride, hydrobromide, hydroiodide, hydroxide, nitrate, nitrosylnitrate and oxalate salts; or a metal chelate.

33. The method of claim 32, wherein the metal precursor is acetylacetonate chelate.

34. The method of claim 29, wherein the metal complex is dried under vacuum at 60° C.

35. The method of claim 29, wherein the metal complex with the nitrogen containing biopolymer is pyrolysed at temperatures ranging from 600° C. to 800° C.

36. The method of claim 29, wherein pyrolysis time ranges from 10 minutes to three hours.

37. The method of claim 36, wherein pyrolysis time ranges from one hour to two hours.

38. The method of claim 29, wherein the nitrogen containing biopolymer-based catalyst comprises metallic or oxidic nickel or cobalt particles, or a combination thereof.

39. The method of claim 29, wherein the nitrogen containing biopolymer-based catalyst comprises from 2 to 100 nitrogen containing carbon layers.

40. The method of claim 39, wherein the nitrogen containing carbon layers comprise graphitic nitrogen, pyridinic nitrogen, or pyrrolic nitrogen, or a combination thereof.
